(19) United States
(12) Patent Application Publication
(10) Pub. No.: US 2026/0243797 A1
(43) Pub. Date: Aug. 20, 2026

(54) METHODS AND SYSTEMS FOR VERIFYING A PHYSICAL ACTIVITY

(71) Applicant: Nicola Gallotti, New York, NY (US)

(72) Inventor: Nicola Gallotti, New York, NY (US)

(21) Appl. No.: 19/450,316

(22) Filed: Jan. 15, 2026

Related U.S. Application Data
(60) Provisional application No. 63/745,650, filed on Jan. 15, 2025.

(51) Int. Cl. G01P 3/00

(52) U.S. Cl. CPC G01P 3/00

(57) ABSTRACT

The present disclosure provides a method of verifying a physical activity. Further, the method may include receiving an activity data from a wearable device system which may be configured to transmit a sensor data over a communication network. Further, the method may include receiving a proximity data from the wearable device system indicative of detection of another wearable device within a proximity range. Further, the method may include analyzing the activity data and the proximity data. Further, the method may include determining a motion similarity metric representing a synchronized movement based on the analyzing. Further, the method may include determining a verification status data based on the motion similarity metric. Further, the method may include storing the verification status data. Further, the method may include transmitting the verification status data to a client system which may be configured to receive verification information.

### REFERENCE TO RELATED APPLICATIONS

This application claims the benefit of U.S. Provisional Patent Application No. 63/745,650, titled “Method and System for Activity Tracking with Integrated Anti-Fraud Detection and Data Synchronization”, filed on Jan. 15, 2025, which is incorporated by reference herein in its entirety.

### FIELD OF DISCLOSURE

The present disclosure relates to the field of data processing. More specifically, the present disclosure relates to methods and systems for verifying a physical activity.

### BACKGROUND

The field of digital physical activity monitoring and data-driven verification technologies has become increasingly important as physical activity data is now widely used in healthcare programs, insurance underwriting, employer wellness initiatives, regulatory compliance frameworks, and incentive-based platforms that rely on objective measurements of human movement. Accurate and trustworthy verification of physical activity plays a critical role in ensuring fairness, accountability, and effectiveness in such programs, particularly when physical activity metrics are tied to financial rewards, benefits eligibility, or compliance determinations.

A desirable objective in the given field is to facilitate reliable verification of physical activity data in a manner that supports scalable deployment, automated evaluation, and downstream consumption by external systems, while maintaining confidence in the authenticity and integrity of the reported activity. Achieving the said objective requires not only the ability to collect activity data but also the capability to assess whether such data accurately represents genuine physical activity performed by an intended participant under legitimate conditions.

However, existing approaches in the given field face a number of technical challenges that limit their ability to achieve the foregoing objective. Many systems rely on activity data generated by individual devices without sufficient contextual evaluation, which may lead to situations where reported activity metrics do not accurately reflect actual physical activity. Such approaches may lack the ability to distinguish between independent activity events and artificially replicated or duplicated movement patterns, thereby undermining confidence in verification outcomes.

In addition, known solutions often struggle to handle the complexity of data generated by heterogeneous devices operating in dynamic physical environments. Differences in sampling rates, timing accuracy, and data formats may introduce inconsistencies that complicate meaningful analysis and comparison of activity data. The said issues are further exacerbated when activity data must be evaluated at scale across large populations, where even small inaccuracies may propagate into significant discrepancies in aggregate results.

Another challenge arises from the limited ability of existing systems to adapt verification logic to varying conditions, usage patterns, or policy requirements. Rigid evaluation criteria may fail to account for contextual factors, leading to either overly permissive outcomes that allow invalid activity to be accepted or overly restrictive outcomes that incorrectly reject legitimate activity. Furthermore, many systems provide limited transparency into how verification decisions are reached, making it difficult to support audit, review, or compliance processes when verification results are questioned.

Scalability and interoperability also present ongoing problems. As activity verification platforms are increasingly integrated with external services, the lack of standardized, verifiable outputs may impede reliable data exchange and downstream processing. Additionally, insufficient mechanisms for preserving decision provenance and data integrity may create challenges in environments where verification outcomes must be trusted over extended periods of time or across organizational boundaries.

Therefore, there is a need for improved methods and systems for verifying a physical activity that may overcome one or more of the preceding problems.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method of verifying a physical activity. Further, the method may include receiving, using a communication device, an activity data from a wearable device system which may be configured to transmit a sensor data over a communication network. Further, the method may include receiving, using the communication device, a proximity data from the wearable device system indicative of detection of another wearable device within a proximity range. Further, the method may include analyzing, using a processing device, the activity data and the proximity data. Further, the method may include determining, using the processing device, a motion similarity metric representing a synchronized movement based on the analyzing. Further, the method may include determining, using the processing device, a verification status data based on the motion similarity metric. Further, the method may include storing, using a storage device, the verification status data. Further, the method may include transmitting, using the communication device, the verification status data to a client system which may be configured to receive verification information.

The present disclosure provides a system for verifying a physical activity. Further, the system may include a communication device. Further, the communication device may be configured for receiving an activity data from a wearable device system which may be configured to transmit a sensor data over a communication network. Further, the communication device may be configured for receiving a proximity data from the wearable device system indicative of detection of another wearable device within a proximity range. Further, the communication device may be configured for transmitting a verification status data to a client system which may be configured to receive verification information. Further, the system may include a processing device communicatively coupled with the communication device. Further, the processing device may be configured for analyzing the activity data and the proximity data. Further, the processing device may be configured for determining a motion similarity metric representing a synchronized movement based on the analyzing. Further, the processing device may be configured for determining the verification status data based on the motion similarity metric. Further, the system may include a storage device communicatively coupled with the processing device. Further, the storage device may be configured for storing the verification status data.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3A illustrates a flowchart of a method 300 of verifying a physical activity, in accordance with some embodiments.

FIG. 3B illustrates a continuation of the flowchart of the method 300 of verifying a physical activity, in accordance with some embodiments.

FIG. 4 illustrates a flowchart of a method 400 of verifying a physical activity including computing, using the processing device 1304, a continuity measure representing a duration consistency of co-location, in accordance with some embodiments.

FIG. 5 illustrates a flowchart of a method 500 of verifying a physical activity including synchronizing, using the processing device 1304, the aligned time windows prior to determining the motion similarity metric, in accordance with some embodiments.

FIG. 6 illustrates a flowchart of a method 600 of verifying a physical activity including generating, using the processing device 1304, a similarity classification result indicative of correlated or non-correlated motion, in accordance with some embodiments.

FIG. 7 illustrates a flowchart of a method 700 of verifying a physical activity including calculating, using the processing device 1304, a fraud risk score representing a likelihood of synchronized non-legitimate activity, in accordance with some embodiments.

FIG. 8 illustrates a flowchart of a method 800 of verifying a physical activity including adjusting, using the processing device 1304, the verification status data, in accordance with some embodiments.

FIG. 9 illustrates a flowchart of a method 900 of verifying a physical activity including identifying, using the processing device 1304, a recurring device association indicative of repeated synchronized movement, in accordance with some embodiments.

FIG. 10 illustrates a flowchart of a method 1000 of verifying a physical activity including modifying, using the processing device 1304, the verification status data to indicate withheld verification, in accordance with some embodiments.

FIG. 11 illustrates a flowchart of a method 1100 of verifying a physical activity including associating, using the processing device 1304, the audit data object with a verification decision identifier, in accordance with some embodiments.

FIG. 12 illustrates a flowchart of a method 1200 of verifying a physical activity including validating, using the processing device 1304, the encoded verification status data against the predefined data schema, in accordance with some embodiments.

FIG. 13 illustrates a block diagram of a system 1300 for verifying a physical activity, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes systems and methods for verifying a physical activity. Further, the verifying of the physical activity includes verifying the physical activity using multi-device sensor correlation. Further, the disclosed system relates generally to physical activity tracking, data verification, and fraud prevention systems. More particularly, the disclosed system concerns an integrated software and hardware platform for certified physical-activity data collection, proximity-based device comparison, and artificial intelligence-based fraud detection suitable for enterprise, healthcare, insurance, and government incentive applications. Further, the disclosed systems may be deployed as a software-as-a-service (SaaS) offering. Further, the disclosed method may be implemented by software deployed as a software-as-a-service (SaaS) offering.

Further, regular physical activity is widely recognized as a cornerstone of preventive healthcare and wellness maintenance. The adoption of fitness trackers, smart wearables, and health monitoring devices has grown significantly. Wearable-based activity tracking is commonly used in consumer applications and enterprise wellness programs, where rewards, incentives, insurance premium adjustments, or other benefits are linked to physical activity performance metrics.

Existing fitness tracking systems, however, exhibit limitations that reduce the said system's suitability for high-stakes, audit-grade applications. Conventional systems typically treat each wearable device as an independent source of data and do not construct a proximity-aware device graph or perform cross-device motion correlation sufficient to detect multiple devices experiencing substantially similar movement patterns.

Further, physical activity monitoring systems generally obtain sensor data from a wearable or mobile device and determine activity occurrence based on predefined rules or thresholds. The said systems may store activity data locally or transmit summaries to a remote system for display or recordkeeping. They primarily rely on single-device data and do not evaluate contemporaneous sensor data from multiple independent devices in combination with proximity-based signals exchanged during an activity event. Additionally, conventional systems do not generate verification outputs supported by evidentiary data suitable for audit or third-party review. As a result, the said systems are limited to recording activity metrics rather than assessing activity authenticity through coordinated, multi-device evaluation.

Further, a significant vulnerability of existing systems is the inability to prevent a single individual from carrying or wearing multiple devices associated with different user accounts. The given limitation allows for potential fraudulent inflation of activity metrics through device stacking, proxy wearing, or mechanical simulation across multiple devices, which may compromise the integrity of incentive programs and create financial exposure for insurers, employers, or government agencies.

Other limitations in the existing systems include the absence of cross-device correlation analysis, lack of proximity-based fraud detection, insufficient authentication mechanisms, inability to generate legally defensible activity certifications, lack of audit trails suitable for regulatory review, and limited behavioral biometric verification.

Further, the combination of rising healthcare costs, broader adoption of value-based care models, and increasing demand for preventive health incentives highlights the need for a fraud-resistant, audit-grade physical activity verification platform capable of supporting large-scale incentive programs with verifiable data integrity.

Further, various prior art systems exist for tracking user activity and providing fitness-related information. For example, U.S. Pat. No. 11,544,591 B2 (Paulina et al.) describes a framework for altering user behavior through motivational state determination. However, the Paulina system focuses primarily on behavioral coaching through contextual awareness and motivational states (preparation, action, maintenance, regress) without addressing the critical security vulnerabilities inherent in multi-device fraud scenarios. Further, the Paulina system assumes data integrity and does not provide mechanisms for cross-device correlation, proximity-based fraud detection, or cryptographic verification of activity data all essential components for high-stakes applications where financial incentives are at risk.

Similarly, U.S. Pat. No. 11,288,142 B2 (Bishop et al.) discloses a recovery strategy for stream processing systems that handles batch processing failures and data replay mechanisms. While Bishop addresses fault tolerance in data processing pipelines, Bishop system does not contemplate the specific challenge of fraudulent activity inflation through device stacking or proxy wearing. Further, the Bishop system is concerned with technical infrastructure reliability rather than activity authenticity verification, and provides no teaching regarding proximity detection, motion signature correlation, or behavioral biometric analysis core innovations of the disclosed system.

Further, stream processing frameworks like Apache Storm™ and Apache Spark™ have been developed to handle high-volume data streams. However, the said systems are designed for general-purpose data processing rather than specialized fraud detection in physical activity tracking contexts. They lack the domain-specific features required for activity verification, such as:
Proximity-aware device graph construction.
Cross-device motion correlation analysis.
Behavioral biometric signature extraction.
Temporal synchronization analysis of activity patterns.
Cryptographically verifiable certification mechanisms.

Further, the disclosed system addresses the said deficiencies by providing a purpose-built system that combines stream processing capabilities with specialized fraud detection algorithms specifically designed for physical activity verification.

Further, consumer fitness platforms such as Fitbit™, Garmin Connect™, and Apple Health™ provide basic activity tracking and goal-setting features. However, the said platforms exhibit several critical limitations that render them unsuitable for audit-grade, incentive-based applications:
Lack of Cross-Device Fraud Detection: Existing platforms treat each wearable device as an independent source of truth and do not construct proximity-aware device graphs or perform cross-device motion correlation sufficient to detect multiple devices experiencing substantially similar movement patterns, creating a significant vulnerability where a single individual may carry or wear multiple devices associated with different user accounts to fraudulently inflate activity metrics.
Absence of Cryptographic Verification: Prior art systems typically provide activity summaries and statistics but do not generate cryptographically signed, tamper-evident certificates suitable for audit or third-party review, preventing insurers, employers, or regulatory agencies to verify the authenticity and integrity of reported activity data with the level of certainty required for financial incentive programs.
Insufficient Authentication Mechanisms: Conventional systems rely primarily on device-level authentication (e.g., user login credentials) without incorporating behavioral biometric verification or continuous authentication through movement patterns, making them vulnerable to device sharing, proxy wearing, and other forms of identity fraud.
Limited Audit Trail Capabilities: Existing platforms do not maintain comprehensive, tamper-evident audit trails with the granularity and security features necessary for regulatory compliance in healthcare and insurance contexts. They lack blockchain anchoring, cryptographic hash chaining, and other mechanisms that would enable independent verification of historical activity records.

Further, recent advancements in machine learning have been applied to activity recognition and behavioral analysis. US Patent Application 2025/0291512 A1 and related applications describe various ML-based approaches for analyzing user behavior and providing recommendations. However, the said systems focus on user experience optimization and personalization rather than fraud detection and data integrity verification.

In some embodiments, the disclosed system distinguishes itself by employing machine learning specifically for fraud detection purposes, utilizing ensemble models (Random Forest, Gradient Boosted Trees, Neural Networks) trained on labeled datasets of confirmed fraudulent and legitimate activity to identify suspicious patterns such as:
Synchronized motion across multiple devices carried by a single individual.
Anomalous step counts exceeding physiologically plausible thresholds.
Temporal inconsistencies indicating mechanical simulation.
Behavioral biometric deviations suggesting device transfer or sharing.

Further, U.S. Pat. No. 11,288,142 B2 (Bishop et al.) provides mechanisms for fault tolerance in stream processing systems through batch replay and state management. While valuable for ensuring system reliability, Bishop does not address the unique challenges of activity data verification where:
1. Batch size modification during replay may lead to over-counting of activity metrics.
2. External data store changes between initial processing and replay may cause inaccurate counter updates.
3. Key elimination in merged datasets may result in misrepresented activity tallies.

Further, the disclosed system recognizes the previously undetected technical problems and provides novel solutions including:
Locking batch composition by persisting current and end event offsets before processing.
Maintaining batch-oriented log segments with keys and corresponding event offsets.
Comparing reprocessing results with persisted results to detect and correct discrepancies.
Generating audit records that identify data inputs and decision factors used in fraud scoring.

Further, the combination of rising healthcare costs, broader adoption of value-based care models, and increasing demand for preventive health incentives highlights several critical technical problems that prior art systems fail to adequately address:

### 1. Multi-Device Fraud Vulnerability

A significant vulnerability of existing systems is the inability to prevent a single individual from carrying or wearing multiple devices associated with different user accounts. The given limitation allows for potential fraudulent inflation of activity metrics through:
   Device stacking (carrying multiple fitness trackers simultaneously).
   Proxy wearing (lending devices to others who perform activities on behalf of the registered user).
   Mechanical simulation across multiple devices using shaker tables or similar equipment.
Further, prior art systems like those described in U.S. Pat. No. 11,544,591 B2 (behavioral coaching) and U.S. Pat. No. 11,288,142 B2 (stream processing) do not contemplate or address the specific fraud vector, which poses substantial financial risks to insurers, employers, and government agencies administering incentive programs.

### 2. Absence of Proximity-Based Fraud Detection

Conventional activity tracking systems do not leverage proximity information to detect suspicious device configurations. They fail to:
   Construct device relationship graphs identifying devices repeatedly detected together.
   Analyze co-location patterns across multiple times and locations.
   Correlate proximity persistence with activity timing to identify fraud attempts.
   Use wireless signals (BLE, UWB, NFC) for real-time proximity verification.
Further, the disclosed system uniquely combines proximity detection with activity verification, recognizing that legitimate users typically do not carry multiple fitness devices simultaneously, whereas fraudsters often exhibit characteristic proximity patterns that may be detected and flagged.

### 3. Insufficient Behavioral Biometric Verification

While U.S. Pat. No. 11,544,591 B2 discusses motivational states and contextual awareness, the said application does not employ behavioral biometric analysis for fraud detection. Prior art systems generally lack:
   Gait signature extraction from accelerometer data.
   Movement pattern consistency verification.
   Cross-device biometric correlation analysis.
   Continuous authentication through activity signatures.
Further, the disclosed system establishes unique movement signatures for each user through parameters such as step duration, stride length, ground contact time, vertical oscillation, and cadence patterns creating personalized gait signatures that may verify activity consistency and detect device transfer or unauthorized use.

### 4. Lack of Audit-Grade Certification Mechanisms

Existing fitness platforms provide activity summaries but not legally defensible certifications. They lack:
   Cryptographically signed certificates with tamper detection.
   Blockchain anchoring for immutable audit trails.
   Certificate revocation mechanisms.
   Third-party verifiable provenance data.
The given limitation renders prior art systems unsuitable for high-stakes applications where activity data must meet evidentiary standards for insurance underwriting, regulatory compliance, or legal proceedings.

### 5. Inadequate Stream Processing Fault Tolerance for Activity Verification

As disclosed in U.S. Pat. No. 11,288,142 B2, general-purpose stream processing systems may experience data integrity issues during batch replay scenarios. However, Bishop does not recognize or address the specific challenges the said issues create for activity tracking applications where:
   Over-counting may result in undeserved incentive payments.
   Under-counting may unfairly penalize legitimate users.
   Inconsistent tallies undermine trust in the verification system.

Further, the combination of rising healthcare costs, broader adoption of value-based care models, and increasing demand for preventive health incentives highlights the need for a fraud-resistant, audit-grade physical activity verification platform capable of supporting large-scale incentive programs with verifiable data integrity.

Further, existing solutions suffer from critical deficiencies that the disclosed system uniquely addresses through:
1. Multi-Device Sensor Correlation: Unlike prior art systems that evaluate activity data from single devices in isolation, the disclosed system correlates data across multiple devices, incorporating environmental and proximity context to determine legitimacy.
2. Proximity-Aware Fraud Detection: The disclosed system constructs device relationship graphs and analyzes co-location patterns to identify suspicious device configurations, a capability entirely absent from prior art fitness tracking systems.
3. Behavioral Biometric Verification: The system establishes and verifies unique movement signatures for each user, providing continuous authentication that prior art systems lack.
4. Cryptographic Certification: The disclosed system generates tamper-evident, cryptographically signed certificates with optional blockchain anchoring providing audit-grade verification unavailable in conventional platforms.
5. Enhanced Stream Processing Integrity: The system addresses previously undetected technical problems in stream processing replay scenarios specifically affecting activity tracking applications.
6. Comprehensive Ecosystem Integration: The disclosed system provides end-to-end functionality spanning mobile applications, backend analytics, heterogeneous device support, enterprise portals, and third-party integration a level of comprehensive integration not achieved in prior art.

Further, the disclosed system represents a significant advancement over prior art through its novel combination of:
Proximity-based multi-device fraud detection using wireless signal analysis.
Cross-device motion correlation employing dynamic time warping and behavioral biometrics.
Machine learning ensemble models specifically trained for activity fraud detection.
Cryptographically verifiable certification with blockchain anchoring.
Activity-specific stream processing safeguards addressing batch replay integrity issues.

Further, no prior art system combines the said elements to provide audit-grade activity verification suitable for high-stakes incentive applications. While individual components (activity tracking, stream processing, machine learning, proximity detection) exist independently in prior art, their integration and specific application to fraud-resistant activity verification represents a non-obvious inventive step that addresses a long-felt but previously unsolved need in the field of preventive health incentive program.

In some embodiments, the present disclosure describes a computer-implemented system and method for verifying physical activity data generated by wearable devices. In particular, the system evaluates multi-device environmental context by detecting proximity and co-movement of multiple wearable devices within a shared physical environment to assess the authenticity of recorded physical activity.

Further, in one aspect, the system receives physical activity data from a primary wearable device associated with a user and detects one or more additional wearable devices located within a predefined distance threshold for a sustained duration. The system performs cross-device motion correlation, temporal synchronization analysis, and baseline deviation evaluation to determine whether the observed activity exhibits patterns indicative of multi-device carry, stacked wearables, or synchronized artificial motion.

Further, a fraud risk score is computed based on aggregated proximity persistence, motion similarity, anomaly detection, and user-specific historical baselines. When the fraud risk score satisfies predetermined verification criteria, the system generates a cryptographically signed activity verification certificate representing validated physical activity. The verification certificate may be configured for consumption by external systems, including reward platforms, insurance underwriting systems, and compliance or audit services.

Further, unlike prior systems that evaluate physical activity data from a single device in isolation, the disclosed system correlates data across multiple devices, incorporating environmental and proximity context to determine legitimacy of recorded activity. In some embodiments, the system further improves data integrity by generating proximity-indexed telemetry records and multi-modal sensor features that are not available in conventional single-device activity tracking architectures.

Further, the disclosed system may include a mobile device executing an application configured to collect physical-activity data from one or more wearable devices; a proximity detection module configured to detect additional wearable devices using wireless protocols such as Bluetooth Low Energy, ultra-wideband, or near-field communication; a backend server configured to receive and process physical-activity and proximity data; a fraud detection engine incorporating machine learning models to compare movement, step patterns, heart rate, and sensor telemetry across multiple devices; and a verification module configured to generate cryptographically signed activity verification certificates.

Further, in some embodiments, the system performs: (i) proximity event logging, (ii) feature extraction from motion and physiological telemetry, (iii) computation of cross-device similarity metrics, and (iv) issuance of a cryptographically verifiable certificate when similarity metrics satisfy non-fraud criteria. Machine learning models classify activity as fraudulent or legitimate using pattern analysis of proximity recurrence, temporal step correlation, movement biometric signatures, statistical anomaly detection, and deviation from user-specific historical activity patterns.

Further, the disclosed system further provides a complete ecosystem, including scalable cloud infrastructure, mobile applications for iOS and Android platforms, device integration supporting heterogeneous wearable devices, an advanced analytics engine with real-time processing capabilities, an enterprise partner portal with compliance reporting, and a comprehensive API framework for third-party integration.

Further, by combining multi-device sensor correlation, proximity-aware analysis, anomaly detection, and cryptographically verifiable certification, the disclosed system provides a comprehensive platform for verifying physical activity with integrated fraud prevention suitable for enterprise, insurance, or regulatory applications.

In some embodiments, the present disclosure describes a physical activity verification platform comprising three principal architectural layers: a Mobile Application Layer, a Backend Services Layer, and a Device Integration Layer. The said components operate in coordination to provide end-to-end functionality for activity tracking, fraud detection, data certification, and enterprise integration.

Further, the platform enables: continuous multi-device activity tracking with sensor fusion; proximity detection and analysis of both registered user devices and unregistered devices in the environment; multi-modal sensor data analysis incorporating accelerometer, gyroscope, heart rate, GPS, and device telemetry; machine learning-based fraud detection with adaptive thresholds; cryptographic certification of verified activity data; enterprise-level audit reporting with compliance documentation; and RESTful API-based integration with third-party systems.

Further, the system is deployable for use by health insurance providers seeking to offer premium discounts based on verified activity; employers implementing corporate wellness programs with monetary incentives; government healthcare agencies administering preventive care reimbursement programs; commercial wellness platforms requiring fraud-resistant activity data; and research institutions conducting population health studies requiring validated physical activity metrics.

Further, the present disclosure describes a mobile application layer encompassed in the disclosed system. Further, the mobile application layer available for iOS and Android platforms, serves as the primary user interface and local data collection point. Key functional modules include:
User Interface Module: Provides graphical interface elements for activity tracking visualization, real-time verification status display, fraud alert notifications, reward balance information, device management screens, and system settings. The interface implements accessibility features compliant with WCAG 2.1 Level AA standards.
Device Registration and Management: Enables users to register wearable devices via Bluetooth pairing, NFC tap-to-pair functionality, or manual device ID entry. The module maintains a local registry of authorized devices associated with the user account, including device type, firmware version, registration timestamp, and last synchronization time.
Proximity Detection Module: Executes periodic scanning operations at configurable intervals (e.g., periodic scanning in foreground and reduced-frequency scanning in background). Scanning protocols include:
   Bluetooth Low Energy (BLE) advertisement scanning with RSSI measurement.
   Ultra-wideband (UWB) ranging when supported by device hardware.
   Near-field communication (NFC) detection for close-proximity scenarios.
   WiFi Direct discovery as a supplementary proximity signal.
Further, the module implements advanced signal filtering algorithms to reduce false positives from transient device encounters, distinguish between stationary and mobile proximity scenarios, and estimate physical distance using path loss models calibrated for body-worn device scenarios.
Activity Data Collection: Interfaces with registered wearable devices to collect: step count data with per-minute granularity; accelerometer time-series data sampled at 50-100 Hz; heart rate measurements; GPS location data (when enabled and permitted); device battery status; and firmware diagnostic data. Data collection operates continuously with battery-optimized protocols including adaptive sampling rates and selective sensor activation.
Local Data Caching: Implements encrypted local storage using platform-native secure storage APIs (iOS Keychain, Android Keystore) for temporary data retention during offline operation. Cache includes pending activity data awaiting synchronization, proximity event logs, device telemetry, and user session state.
Synchronization Engine: Manages bidirectional data synchronization with backend services using resilient protocols with automatic retry logic, exponential backoff for failed transmissions, data compression to minimize bandwidth consumption, and delta synchronization to transmit only changed data. Synchronization triggers include: periodic intervals (every 15-60 minutes), significant activity accumulation thresholds, proximity event detection, and user-initiated manual sync requests.
Geofencing Enforcement: For programs requiring regional operation restrictions, implements geofencing to verify user location falls within authorized geographic boundaries. Uses GPS and network-based location with fallback mechanisms. Non-compliant location detection results in activity data flagging or temporary service suspension based on program policy.
Background Processing: Utilizes platform background execution APIs to maintain proximity scanning, activity collection, and data synchronization when application is not in foreground. Implements battery optimization strategies including work scheduling, reduced scanning frequency, and selective sensor activation to balance functionality with power consumption.
Security Module: Implements certificate pinning for API communications, local data encryption using AES-256, biometric authentication for app access (fingerprint, face recognition), and secure credential storage. Includes anti-tampering detection monitoring for root access, debugger attachment, and emulator execution.

Further, the present disclosure describes the backend services layer associated with the disclosed system. Further, the backend infrastructure, deployed on cloud platforms (AWS, Azure, or GCP) with multi-region redundancy, comprises:
User Account Management Service: Handles user registration, authentication, and authorization using multi-factor authentication (MFA) with TOTP or SMS-based verification. Implements role-based access control (RBAC) supporting user roles (end-users, program administrators, enterprise partners, system administrators) with granular permission management. Supports organizational account structures for enterprise deployments with hierarchical user management and bulk provisioning capabilities.
Activity Data Storage System: Utilizes hybrid database architecture:
   Relational database (PostgreSQL) for user profiles, account information, device registry, and program enrollment data.
   NoSQL time-series database (InfluxDB or TimescaleDB) for high-volume activity telemetry with automatic data retention policies.
   Object storage (S3-compatible) for raw sensor data archives and audit documentation.
   In-memory cache (Redis) for session management and real-time analytics.
Further, data retention policies include: 90-day hot storage for active analysis, 2-year warm storage for historical reference, and 7-year cold archive for regulatory compliance (configurable per jurisdiction requirements).
Fraud Detection Service: Implements a multi-stage fraud analysis pipeline:
   Stage 1—Proximity Analysis: Evaluates device co-location patterns from BLE/UWB proximity events. Constructs device relationship graphs identifying devices repeatedly detected together. Calculates proximity frequency scores, duration metrics, and movement correlation coefficients.
   Stage 2—Movement Signature Analysis: Extracts behavioral biometric features from accelerometer data, including gait patterns, cadence consistency, stride length distribution, and movement intensity profiles. Compares signatures across devices using dynamic time warping (DTW) algorithms to identify abnormally high correlation indicative of devices carried by same individual.
   Stage 3—Anomaly Detection: Applies statistical methods and machine learning models (Isolation Forest, Auto encoder neural networks) to identify:
      a. Step count values exceeding physiologically plausible thresholds.
      b. Activity patterns inconsistent with user historical norms.
      c. Sudden unexplained changes in activity levels.
      d. Implausible temporal patterns (e.g., high-intensity activity during typical sleep hours).
   Stage 4—Cross-Device Synchronization Analysis: Performs time-series correlation analysis comparing step count trajectories, activity timing, and sensor data patterns across devices detected in proximity. High correlation coefficients combined with frequent proximity events trigger fraud alerts.
   Stage 5—Machine Learning Classification: Employs ensemble models (such as Random Forest, Gradient Boosted Trees, and other available models in the future) trained on labeled datasets of confirmed fraudulent and legitimate activity. Features include: proximity frequency, movement correlation scores, anomaly scores, historical pattern deviation, device diversity metrics, and temporal consistency measures. Model outputs probability scores for fraud likelihood with confidence intervals.
   Stage 6—Adaptive Thresholding: Implements dynamic decision thresholds adjusting based on: individual user baseline patterns, population-level statistical distributions, program-specific risk tolerance, and temporal factors (e.g., higher thresholds during initial user onboarding period).
Further, the fraud detection engine processes data in near-real-time (latency <5 minutes from data ingestion to fraud classification) and batch modes (comprehensive daily analysis of accumulated data).
Activity Certification Module: Generates verifiable certificates for validated activity data containing:
   User identification (anonymized or personally identified based on program requirements).
   Activity period (date range).
   Verified step counts and activity metrics.
   Fraud risk score and confidence level.
   Data collection methods and devices used.
   Cryptographic digital signature (RSA-2048 or ECDSA).
   Timestamp of certification.
   Unique certificate identifier.
   Optional blockchain transaction hash for immutable audit trail.
Further, certificates are generated in multiple formats (JSON, XML, PDF) and made available via API and user portal. Blockchain anchoring uses public or consortium blockchains (Ethereum, Hyperledger) storing certificate hashes for tamper-evident verification.
Reward Processing Engine: Manages incentive calculation and distribution:
   Reward rule engine supporting complex policy definitions (step thresholds, activity type bonuses, consistency rewards, challenge completion bonuses).
   Personalized reward tier assignment based on user health profiles, physical limitations, chronic conditions, and historical performance.
   Dynamic incentive adjustment algorithms optimizing reward structures for sustained engagement.
   Integration with payment systems (ACH transfer, digital wallets, gift cards, insurance premium adjustments).
   Tax reporting compliance (1099 generation for US programs).
   Partner-specific reimbursement rule implementation.
Further, processing operates on configurable cycles (daily, weekly, monthly) with fraud-verified activity data as input. Failed fraud verification results in reward withholding with user notification.
Enterprise API Framework: Provides RESTful and GraphQL APIs with:
   Comprehensive endpoint documentation (OpenAPI/Swagger specifications).
   OAuth 2.0 authentication with JWT tokens.
   Rate limiting and quota management.
   Webhook support for event-driven integrations.
   Bulk data retrieval operations with pagination.
   Real-time activity streaming endpoints.
   Certificate verification endpoints for third-party validation.
   Administrative functions for program configuration.
Real-Time Data Processing Pipeline: Utilizes stream processing frameworks (Apache Kafka, AWS Kinesis) for:
   Ingestion of high-volume activity telemetry from mobile applications.
   Real-time fraud detection with immediate alert generation.
   Live dashboard updates for enterprise partners.
   Event-driven workflows triggering on specific activity patterns.
   Data enrichment and transformation before storage.
Analytics and Reporting Service: Generates insights for multiple stakeholder types:
   User-facing: personal activity trends, progress toward goals, comparative benchmarks.
   Enterprise partners: program participation metrics, ROI analysis, population health indicators, fraud detection statistics.
   System administrators: platform performance metrics, data quality reports, fraud detection model performance.
   Auditors: compliance reports, audit trail exports, data provenance documentation.
Partner Portal: Web-based interface for enterprise users providing:
   Real-time program performance dashboards with customizable metrics.
   User enrollment and management tools.
   Reward policy configuration interfaces.
   Fraud alert review and investigation tools.
   Audit log access with advanced filtering and export capabilities.
   Compliance documentation generation.
   API key management and usage monitoring.
   Support ticket system with SLA tracking.
Compliance and Security Services: Implements:
   HIPAA compliance for health data handling (for US healthcare applications).
   GDPR compliance for EU user data protection.
   SOC 2 Type II controls for data security and availability.
   Encryption at rest (AES-256) and in transit (TLS 1.3).
   Comprehensive audit logging with tamper-evident log storage.
   Regular security vulnerability scanning and penetration testing.
   Data breach response procedures.
   Privacy-preserving analytics using differential privacy techniques.

Further, the present disclosure describes a device integration layer associated with the disclosed system. Further, the device integration layer provides abstraction and integration capabilities for heterogeneous wearable devices:
Bluetooth Connectivity Manager: Implements BLE Central role for connection management including: device discovery and filtering, connection establishment with automatic retry, connection monitoring with reconnection logic, and GATT service enumeration and interaction.
Multi-Device Support Framework: Supports device categories, including:
   Smartwatches (Apple Watch, Wear OS, Fitbit, Garmin, Samsung Galaxy Watch).
   Fitness bands (Fitbit, Xiaomi Mi Band, WHOOP, Oura Ring).
   Smart clothing (Athos, Sensoria, Hexoskin).
   Shoe-mounted sensors (RunScribe, Stryd, Garmin Running Dynamics Pod).
   Chest strap heart rate monitors (Polar, Garmin, Wahoo).
   Smart rings (Oura, Motiv, Circular).
   Medical-grade wearables (AliveCor, BioIntelliSense, VitalConnect).\
Further, each device type includes type-specific driver modules implementing data collection protocols, firmware update capabilities, and device-specific features.
Data Normalization Engine: Converts heterogeneous device data formats into standardized internal representations:
   Step count normalization accounting for device-specific counting algorithms.
   Accelerometer data resampling and unit conversion.
   Heart rate zone mapping to standardized intensity levels.
   Activity type classification standardization.
   Timestamp synchronization handling timezone and clock drift issues.
Third-Party API Integration: Supports data import from major fitness platforms:
   Apple HealthKit integration for iOS devices.
   Google Fit integration for Android devices.
   Fitbit Web API.
   Garmin Connect API.
   Strava API.
   Under Armour Connected Fitness API.
Integration handles OAuth authorization flows, data synchronization scheduling, incremental data updates, and API rate limit management.
Device Telemetry Processing: Captures and analyzes device health metrics:
   Battery level and charging patterns.
   Firmware version tracking.
   Connection stability metrics.
   Data transmission success rates.
   Sensor calibration status.
   Device error logs.
Telemetry data informs fraud detection (e.g., battery patterns inconsistent with claimed usage) and enables proactive device issue detection.

Further, the present disclosure describes a proximity detection system associated with the disclosed system. Further, the proximity detection system represents a novel component enabling identification of potentially fraudulent device configurations through environmental device awareness. Further, the detection technologies include:
Bluetooth Low Energy (BLE) Detection:
Primary proximity detection mechanism utilizing BLE advertisement scanning. Mobile application continuously or periodically scans for BLE advertisements from nearby devices. Detection process includes:
   Advertisement Parsing: Extracts device identifiers (MAC addresses, UUIDs), manufacturer data, service UUIDs, and advertising flags. Identifies wearable devices based on known manufacturer identifiers and service profiles.
   RSSI-Based Distance Estimation: Received Signal Strength Indicator (RSSI) values converted to estimated physical distance using path loss models. Calibration accounts for body absorption, antenna orientation, and environmental multipath effects. Distance estimation may be derived from RSSI and calibration models, and may be represented as an estimated range and/or confidence value.
   Signal Filtering: Implements Kalman filtering or moving average smoothing to reduce RSSI variability. Distinguishes between direct path and reflected signals. Temporal consistency analysis identifies stable versus transient proximity.
Ultra-Wideband (UWB) Ranging:
For devices supporting UWB (iPhone 11+, Samsung Galaxy Note 20+, compatible UWB-enabled wearables), provides centimeter-level precision distance measurements using time-of-flight calculations. UWB ranging resistant to multipath interference and provides angle-of-arrival information enabling device position estimation.
Near-Field Communication (NFC):
Detects immediate physical proximity (<10 cm) useful for intentional device verification scenarios and close-contact fraud detection (e.g., wallet full of devices).
Wifi Direct and Wifi Aware:
Supplementary proximity signals on supported platforms providing device discovery and ranging within WiFi operational ranges (typically 50-100 meters).

Further, each detected proximity event is characterized by:
Device Identifiers: MAC addresses, unique device IDs, device type/model.
Timestamp: Event detection time with millisecond precision.
Distance Estimate: Physical separation estimate with confidence interval.
Duration: Length of proximity encounter (for persistent connections).
RSSI Profile: Signal strength measurements over time.
Motion Context: User movement state during proximity event (stationary, walking, running, vehicle transport).
Location Context: Geographic location if GPS enabled.
Device Relationship Classification: New device, previously seen device, frequently encountered device, registered device belonging to different user account.

Further, historical proximity data is analyzed to identify fraud-indicative patterns:
Persistent Co-Location: Devices consistently detected together across multiple times and locations indicate potential device-carrying fraud. Statistical measures include:
   Co-location frequency (percentage of time windows with simultaneous detection).
   Co-location consistency (standard deviation of inter-detection intervals).
   Geographic diversity of co-location events (number of distinct locations).
Synchronized Movement Patterns: Accelerometer data from proximate devices compared using correlation analysis and dynamic time warping. High correlation (>0.85) indicates devices experiencing identical motion profiles consistent with being carried by single individual or attached to same carrier object.
Relationship Graph Analysis: Constructs graph structure with devices as nodes and proximity relationships as edges weighted by encounter frequency and correlation strength. Graph analysis algorithms (community detection, centrality measures) identify suspicious device clusters and recurring patterns.
Temporal Pattern Analysis: Examines timing of proximity events relative to activity patterns. For example, proximity occurring primarily during high-activity periods suggests fraud targeting incentive thresholds.

Further, proximity detection implements the following privacy safeguards:
Device identifiers stored in hashed form preventing direct identification.
User devices from same enterprise program flagged without individual identification.
Proximity detection may be temporarily disabled in privacy-sensitive contexts.
Users notified when their device detected by other platform users.
Aggregated proximity statistics prevent individual tracking.

Further, the present disclosure describes a fraud detection engine associated with the disclosed system. Further, the fraud detection engine combines multiple analytical techniques in a layered approach providing comprehensive fraud risk assessment:
Machine Learning Models
   a. Training Data: Models trained on labeled datasets, including:
      Known fraudulent cases from pilot programs and red team testing.
      Confirmed legitimate activity from validated users.
      Synthetic fraud scenarios generated through simulation.
      Crowd-sourced fraud attempt data with user consent.
   Training datasets include a minimum of 100,000 labeled activity sessions with balanced representation of fraud types.
   b. Feature Engineering: Extracts hundreds of features across categories:
   Proximity Features:
      Number of distinct devices detected in proximity.
      Frequency of proximity events.
      Average duration of proximity encounters.
      Number of devices with high movement correlation.
      Device diversity scores.
      Proximity recurrence patterns.
   Movement Features:
      Step count statistical distributions (mean, variance, skewness, kurtosis).
      Cadence consistency metrics.
      Activity intensity profiles.
      Gait pattern features (step regularity, symmetry, variability).
      Activity type diversity.
      Temporal activity patterns.
   Anomaly Features:
      Deviation from user historical means.
      Deviation from population distributions.
      Sudden pattern changes.
      Implausible activity levels.
      Consistency violations.
   Device Features:
      Number of registered devices.
      Device switching frequency.
      Device type patterns.
      Battery usage patterns.
      Connection stability metrics.
   Behavioral Features:
      Response to verification challenges.
      App engagement patterns.
      Reward claiming behaviors.
      Historical fraud risk scores.
   c. Model Architecture: Employs ensemble approach combining:
      Random Forest: Handles non-linear feature interactions, resistant to overfitting, provides feature importance rankings.
      Gradient Boosted Trees (XGBoost): Optimizes prediction accuracy through sequential learning.
      Neural Networks: Deep learning models for complex pattern recognition in time-series sensor data.
      Logistic Regression: Baseline interpretable model providing probability calibration.
   Ensemble predictions combined using weighted voting or stacking approaches. Model outputs calibrated probability scores ranging 0-1 indicating fraud likelihood.
   Model outputs may be calibrated and evaluated using one or more performance measures.
   d. Continuous Learning: Models retrained periodically with:
      Newly labeled fraud cases.
      Confirmed false positives/negatives from human review.
      Emerging fraud pattern detection through unsupervised learning.
      A/B testing of model variants with champion/challenger framework.
Anomaly Detection Algorithms
Complement supervised learning with unsupervised anomaly detection:
   a. Statistical Outlier Detection:
      Z-score analysis identifying values exceeding 3-4 standard deviations from mean.
      Interquartile range (IQR) methods flagging extreme outliers.
      Population-based percentile thresholds.
   b. Isolation Forest:
   Unsupervised machine learning identifying anomalies through random partitioning. Effective for high-dimensional data with unknown fraud patterns.
   c. Autoencoder Neural Networks:
   Train on legitimate activity patterns to learn compressed representations. Reconstruction error serves as anomaly score-fraudulent patterns poorly reconstructed due to deviation from learned normal patterns.
   d. Time-Series Anomaly Detection
      Change point detection identifying sudden distribution shifts.
      Seasonal decomposition separating trends from anomalies.
      ARIMA residual analysis.
      LSTM-based sequence anomaly detection.
Cross-Device Correlation Analysis
Compares activity data across devices detected in proximity:
   a. Step Count Correlation: Pearson and Spearman correlation coefficients calculated for step count time-series. High correlation (>0.8) across extended periods indicates synchronized counting.
   b. Activity Timing Correlation: Measures temporal alignment of activity periods. Identical start/stop times across devices suggest device carrying rather than independent wearing.
   c. Accelerometer Pattern Matching:
      Cross-correlation of raw accelerometer signals.
      Dynamic Time Warping (DTW) distance metrics allowing temporal shifts.
      Frequency domain analysis comparing power spectral densities.
      Wavelet-based feature comparison.
   d. Heart Rate Pattern Analysis: For devices with heart rate sensors, compares cardiovascular response patterns. Legitimate independent users show uncorrelated heart rate patterns, while carried devices may show identical or absent heart rate data.
Behavioral Biometric Analysis
Establishes unique movement signatures for each user:
   a. Gait Analysis: Extracts gait parameters from accelerometer data:
      Step duration and regularity.
      Stride length estimates.
      Ground contact time.
      Vertical oscillation.
      Cadence patterns.
      Asymmetry measures.
   Creates personalized gait signature used to verify activity consistency and detect device transfer or wearing by different individual.
   b. Activity Pattern Profiling: Learns user-specific patterns:
      Typical activity times and durations.
      Activity intensity preferences.
      Activity type preferences.
      recovery patterns.
      Weekly and seasonal patterns.
   Significant deviations trigger fraud alerts.
   c. Behavioral Authentication: Continuous authentication through movement patterns supplements traditional authentication methods. Activity data inconsistent with established behavioral biometrics indicates potential account compromise or device misuse.
Confidence Scoring and Decision Making
Fraud detection outputs risk scores with confidence levels:
   a. Risk Scores: Continuous scale 0-100 where:
      0-20: Very low fraud risk (high confidence legitimate)
      21-40: Low fraud risk
      41-60: Moderate fraud risk (requires additional scrutiny)
      61-80: High fraud risk
      81-100: Very high fraud risk (high confidence fraudulent)
   b. Decision Thresholds: Configurable per program requirements:
      Conservative: Threshold 70+ for fraud flag (minimize false accusations, accept some fraud).
      Balanced: Threshold 60+ for fraud flag.
      Aggressive: Threshold 50+ for fraud flag (maximize fraud detection, accept some false positives).
   c. Confidence Intervals: Each risk score includes confidence interval indicating prediction uncertainty. Low confidence scores trigger human review or additional verification procedures.
   d. Explainability: Fraud decisions accompanied by explanations identifying primary contributing factors (e.g., “High correlation with 3 nearby devices detected in 15 of 20 activity sessions”). Explainability supports user appeals and regulatory compliance.
Adaptive Threshold:
Fraud detection thresholds adapt based on:
   a. User Tenure: New users subject to more lenient thresholds during initial weeks, allowing system to establish baseline patterns. Thresholds tighten as historical data accumulates.
   b. Program Context: Different programs have different fraud risk tolerances:
      High-value insurance premium programs: strict thresholds.
      Low-value wellness challenges: moderate thresholds.
      Research studies: prioritize data quality over strict fraud prevention.
   c. Population Statistics: Thresholds calibrated based on observed fraud rates in similar user populations, geographic regions, and demographic segments.
   d. Temporal Factors: Adjusted for seasonal variations, holidays, special events that may create legitimate anomalies.
Geolocation and Temporal Plausibility
Additional fraud detection layers:
   Impossible Travel Detection: Flags activity data reporting locations separated by distances impossible to traverse in reported time intervals.
   Activity Plausibility: Validates activity intensity and duration against physiological limits, considering user age, health status, and historical performance.
   Environmental Context: Cross-references activity with weather data, terrain information, and infrastructure availability to validate plausibility (e.g., outdoor running activity during severe weather warnings flagged for review).
Fraud Reporting and Response
Upon fraud detection:
   a. Immediate Actions:
      Activity verification withheld pending investigation.
      Reward issuance suspended.
      User notification of verification failure (with explanation when appropriate).
      Fraud incident logged with all supporting evidence.
   b. Investigation Workflow:
      Cases above threshold automatically escalated to fraud investigation team.
      Analysts review evidence including proximity logs, correlation analysis, timeline reconstruction.
      User contact initiated for explanation when appropriate.
      Decision rendered: fraud confirmed, legitimate activity verified, or inconclusive requiring additional monitoring.
   c. User Appeals:
      Users may appeal fraud determinations.
      Appeal submission includes user explanation and supporting evidence.
      Human review of original fraud detection evidence and user appeal.
      Feedback loop updates machine learning models with appeal outcomes.
   d. Enforcement Actions (for confirmed fraud):
      Account suspension or termination.
      Recovery of improperly awarded rewards.
      Reporting to partner organizations (employers, insurers).
      Legal action in severe cases involving terms of service violations.

Further, the present disclosure describes activity verification and certification relative to the disclosed system. Further, verified legitimate activity results in a cryptographically secured certification suitable for audit and third-party validation.
Certification Generation Process
   a. Data Aggregation: Collects all verified activity data for certification period, including:
      Daily step counts with hourly breakdowns.
      Activity type classifications (walking, running, cycling, etc.).
      Total active minutes.
      Distance estimates.
      Calorie expenditure calculations.
      Heart rate zone distributions (when available).
      Device sources for all data points.
   b. Fraud Risk Assessment: Includes final fraud risk score and confidence level for the certified period. Certificates are issued only for an activity that passes fraud detection thresholds.
   c. Metadata Compilation: Assembles certification metadata:
      User identification (personal or anonymized per program requirements).
      Certification period (start/end dates and times).
      Certification timestamp.
      Issuing platform version.
      Applicable program identifiers.
      Data quality indicators.
   d. Cryptographic Signing: Certificate content serialized and signed using:
      RSA-2048 or RSA-4096 digital signatures.
      ECDSA with P-256 or P-384 curves.
      EdDSA (Ed25519) for modern high-security applications.
   Private keys are stored in Hardware Security Modules (HSMs) with strict access controls. Public keys are published for certificate verification.
   e. Certificate Issuance: Certificates made available through:
      User portal download (PDF and JSON formats).
      API endpoints for programmatic retrieval.
      Email delivery to user and authorized parties.
      Partner portal access for employers/insurers.
   In some embodiments, the certificate is a structured data object comprising (i) activity metrics, (ii) provenance metadata identifying device sources, (iii) a computed fraud risk score and/or explanation features, and (iv) a digital signature verifiable using a published public key.
   f. Blockchain Anchoring: Certificate hash stored on blockchain providing:
      Tamper-evident record.
      Timestamp proof.
      Immutable audit trail.
      Third-party verifiable existence proof.
   Supported blockchains include public chains (Ethereum mainnet or L2s) or permissioned consortium chains (Hyperledger Fabric).
Certificate Validation
Third parties validate certificates through:
   Signature Verification: Using published public keys to verify cryptographic signatures, confirming certificate authenticity and detecting any tampering.
   Blockchain Verification: For blockchain-anchored certificates, verifiers query blockchain to confirm certificate hash exists at claimed timestamp, providing independent validation of certificate issuance.
   Revocation Check: Validation process includes checking certificate revocation lists for withdrawn certifications due to subsequent fraud discovery or user request.

Further, the present disclosure describes the reward system architecture associated with the disclosed system. Further, the integrated reward processing system incentivizes sustained physical activity while maintaining fraud resistance.
Reward Calculation Engine
   a. Base Reward Formula: Calculates rewards using verified activity metrics:
      Step-based rewards (e.g., $0.01 per 1,000 verified steps).
      Activity-type multipliers (running 1.5×, cycling 1.3×, walking 1.0×).
      Intensity bonuses for elevated heart rate zones.
      Consistency bonuses for meeting daily goals.
      Challenge completion bonuses.
   b. Personalized Tier System: Adjusts reward structures based on individual capabilities:
      Medical condition accommodations (reduced thresholds for chronic conditions).
      Age-adjusted targets.
      Disability-inclusive metrics.
      Progressive goal systems adapting to user improvement.
   c. Dynamic Incentive Optimization: Machine learning models optimize reward structures to:
      Maximize sustained engagement.
      Balance cost-effectiveness with participation.
      Adapt to seasonal and demographic patterns.
      Test alternative incentive structures through A/B testing.
Reward Distribution
   a. Payment Methods:
      Direct deposit (ACH transfer).
      Digital wallets (PayPal, Venmo).
      Gift card issuance.
      Insurance premium credits.
      Employer payroll integration.
      Cryptocurrency payments (where legally permissible).
   b. Processing Cycles: Configurable processing schedules (daily, weekly, monthly) with fraud verification gate before payment authorization.
   c. Tax Compliance: Automated 1099 generation for U.S. users exceeding annual thresholds, tax withholding when required, and international tax documentation support.
Fraud Impact on Rewards
   Immediate Withholding: Rewards automatically withheld for activity failing fraud verification until the investigation is complete.
   Retroactive Recovery: Previously distributed rewards clawed back upon confirmed fraud discovery, with account balance adjustments and payment recovery procedures.
   Suspension Protocols: Repeat fraud attempts result in temporary or permanent reward program suspension while maintaining platform access for legitimate activity tracking.

Further, the present disclosure describes an enterprise integration framework associated with the disclosed system:
API Architecture
   a. RESTful Endpoints
      ‘/api/v1/users’-User management operations.
      ‘/api/v1/activity’-Activity data retrieval.
      ‘/api/v1/certificates’-Certificate generation and validation.
      ‘/api/v1/rewards’-Reward balance and transaction queries.
      ‘/api/v1/programs’-Program configuration management.
      ‘/api/v1/analytics’-Aggregated reporting data.
   b. GraphQL Interface: Flexible query interface enabling partners to request precisely needed data with single API calls, reducing network overhead and improving integration efficiency.
   c. Webhook System: Event-driven notifications for:
      Activity verification completions.
      Fraud detection alerts.
      Reward distributions.
      Certificate generations.
      User enrollment/departures.
      System anomalies.
Partner Integration Types
   a. Insurance Provider Integration:
      Policy holder verification.
      Premium adjustment calculation feeds.
      Claims data integration.
      Risk assessment reporting.
      Compliance documentation generation.
   b. Employer Wellness Integration:
      HR system synchronization (Workday, ADP, SAP SuccessFactors).
      Employee roster management.
      Wellness program enrollment automation.
      ROI reporting dashboards.
      Benefits administration integration.
   c. Government Healthcare Integration:
      Medicare/Medicaid system integration.
      Preventive care reimbursement processing.
      Population health reporting.
      HIPAA-compliant data exchange.
      Electronic health record (EHR) integration.
   d. Third-Party Wellness Platforms:
      Data import/export with major fitness platforms.
      White-label integration capabilities.
      Co-branded application support.
      Unified user experience across platforms.
Analytics and Reporting
   a. Real-Time Dashboards: Web-based interfaces displaying:
      Current participation rates.
      Aggregate activity metrics.
      Fraud detection statistics.
      Reward program costs.
      User engagement trends.
      Device usage distributions.
   b. Custom Report Generation: Scheduled or on-demand reports, including:
      Executive summaries for stakeholders.
      Detailed activity breakdowns by demographic segments.
      Fraud analysis reports.
      ROI calculations and projections.
      Compliance audit documentation.
      Population health trend analysis.
   c. Predictive Analytics: Machine learning models forecasting:
      Future participation trends.
      Expected reward program costs.
      Fraud rate projections.
      User retention probabilities.
      Program effectiveness predictions.

Further, the present disclosure describes security and compliance of the disclosed system:
Data Security Measures
   a. Encryption Standards:
      AES-256 encryption for data at rest.
      TLS 1.3 for data in transit.
      End-to-end encryption for sensitive PII.
      Key rotation policies (90-day cycles).
      HSM-protected cryptographic keys.
   b. Access Controls:
      Multi-factor authentication is mandatory for all administrative access.
      Role-based access control with the principle of least privilege.
      Session management with automatic timeouts.
      IP whitelisting for partner API access.
      Comprehensive audit logging of all data access.
   c. Network Security:
      Web application firewall (WAF).
      DDoS protection.
      Intrusion detection/prevention systems.
      Regular penetration testing.
      Bug bounty program.
Regulatory Compliance
   a. HIPAA Compliance (U.S. Healthcare):
      Business Associate Agreements with partners.
      Protected Health Information (PHI) handling protocols.
      Breach notification procedures.
      Regular compliance audits.
      Staff training programs.
   b. GDPR Compliance (European Union):
      Data subject rights implementation (access, rectification, erasure, portability).
      Consent management systems.
      Privacy by design architecture.
      Data Protection Impact Assessments.
      EU representative designation.
   c. CCPA Compliance (California):
      Consumer rights request handling.
      Do Not Sell disclosures.
      Privacy policy transparency.
      Opt-out mechanisms.
   d. SOC 2 Type II: Annual independent audits verifying security, availability, processing integrity, confidentiality, and privacy controls.
   e. Industry-Specific Regulations:
      FDA medical device regulations (if applicable).
      FTC advertising and endorsement guidelines.
      State insurance regulations.
      International data transfer regulations.

Further, the present disclosure describes scalability and performance of the disclosed system:
System Capacity
The system may be implemented using horizontally scalable services and distributed storage.
Infrastructure Architecture
   a. Microservices Design: Independently scalable services for authentication, activity processing, fraud detection, certification, rewards, and analytics.
   b. Auto-Scaling: Dynamic resource allocation based on load with horizontal scaling for compute resources and vertical scaling for database instances.
   c. Geographic Distribution: Multi-region deployment with:
      Edge caching for mobile API endpoints.
      Regional data residency compliance.
      Active-active failover configurations.
      CDN distribution for static assets.
   d. Database Optimization:
      Read replicas for query distribution.
      Sharding strategies for large datasets.
      Indexing optimization.
      Query caching layers.

Further, the present disclosure describes the following user experience features:
Mobile Application Features
   a. Activity Visualization:
      Real-time step counters.
      Daily/weekly/monthly trend charts.
      Activity heatmaps showing patterns.
      Comparative benchmarks against personal goals and population averages.
      Achievement badges and milestone celebrations.
   b. Social Features:
      Optional friend connections and activity sharing.
      Team challenges and competitions.
      Leaderboards with privacy controls.
      Encouragement messaging.
      Community forums.
   c. Gamification:
      Progressive achievement systems.
      Challenge completion rewards.
      Streak tracking.
      Virtual trophies and collections.
      Seasonal events and limited-time challenges.
   d. Health Integration:
      Integration with nutrition tracking.
      Sleep quality correlation analysis.
      Weight management tracking.
      Heart health metrics.
      Wellness goal setting.
Notifications and Engagement
   a. Push Notifications:
      Daily activity reminders.
      Goal achievement celebrations.
      Challenge participation invitations.
      Verification status updates.
      Reward distribution notifications.
   b. In-App Messaging: Personalized messages based on activity patterns, encouraging increased participation during low-activity periods.
   c. Email Communications: Weekly summary reports, monthly achievements, program updates, and reward statements.

Further, the present disclosure describes the following advanced features and future enhancements associated with the disclosed system:
Planned Enhancements
   a. Computer Vision Integration: Photo-based activity verification requiring users to submit photos during activities, with AI analyzing images for authenticity indicators.
   b. Biometric Liveness Detection: Enhanced fraud prevention through continuous biometric verification including heart rate variability patterns, skin temperature, and galvanic skin response.
   c. Environmental Sensor Fusion: Integration of air quality, temperature, altitude, and weather data for enhanced activity validation and contextual insights.
   d. Social Proof Mechanisms: Optional witness verification where other platform users confirm activity observations, creating distributed fraud prevention.
   e. Advanced Analytics: Predictive health modeling, injury risk assessment, personalized training recommendations, and longitudinal health outcome tracking.
Research and Development
Ongoing research initiatives include:
   Quantum-resistant cryptographic algorithms for future-proof certificate security.
   Federated learning approaches enabling privacy-preserving machine learning model training.
   Edge computing implementations for reduced latency fraud detection.
   Novel proximity detection methods using acoustic fingerprinting and ambient light sensors.
   Integration with emerging wearable technologies (smart contact lenses, subcutaneous monitors, smart tattoos).

In some embodiments, the present disclosure describes a system for verifying physical activity. Further, the system comprises a mobile computing device executing an application configured to obtain physical-activity telemetry from at least one wearable device associated with a user. Further, the system comprises a proximity module configured to detect wireless advertisements from one or more additional wearable devices within a proximity range and to generate proximity event records, each proximity event record comprising at least a device identifier, a timestamp, and a proximity indicator. Further, the system comprises a server system configured to receive the physical-activity telemetry and the proximity event records and to store the proximity event records in a tamper-evident log. Further, the system comprises a feature extraction module configured to derive one or more motion features from the physical-activity telemetry. Further, the system comprises a cross-device analysis module configured to compute, for at least one-time window, one or more similarity metrics between (i) motion features associated with the wearable device associated with the user and (ii) motion features associated with at least one additional wearable device repeatedly detected within the proximity range. Further, the system comprises a decision module configured to generate a fraud risk score using at least the one or more similarity metrics and a proximity recurrence measure indicative of repeated co-location over time, and to determine whether the physical-activity telemetry is indicative of activity performed by a single user. Further, the system comprises a certificate module configured, responsive to the decision module determining that a non-fraud condition is satisfied, to generate a cryptographically verifiable activity certificate, and otherwise to withhold issuance of the activity certificate.

In some embodiments, the present disclosure describes a method of verifying physical activity. Further, the method may include a step of collecting, via a mobile application executing on a mobile computing device, physical-activity telemetry from a first wearable device associated with a user. Further, the method may include a step of detecting, via a proximity detection module, one or more additional wearable devices within a proximity range using wireless communication signals. Further, the method may include a step of generating proximity event records comprising device identifier data, timestamps, and proximity indicators for the one or more additional wearable devices. Further, the method may include a step of transmitting the physical-activity telemetry and the proximity event records to a backend server. Further, the method may include a step of computing, via a fraud detection engine executing on the backend server, at least one cross-device similarity metric based on a temporally aligned comparison between (i) movement-related sensor data of the first wearable device and (ii) movement-related sensor data associated with at least one additional wearable device detected within the proximity range. Further, the method may include a step of generating a fraud risk score based at least in part on the at least one cross-device similarity metric and a proximity recurrence measure derived from the proximity event records. Further, the method may include a step of determining whether the physical-activity telemetry is legitimate by comparing the fraud risk score to a decision criterion. Further, the method may include a step of generating, when the physical-activity telemetry is determined to be legitimate, a cryptographically verifiable activity certificate and providing the cryptographically verifiable activity certificate to an external system.

In some embodiments, the present disclosure describes a non-transitory computer-readable medium storing instructions that, when executed by one or more processors of a computing system, cause the processors to perform operations. Further, the operations may include:
Detecting one or more wearable devices in proximity to a mobile computing device using one or more wireless communication signals.
Receiving physical-activity telemetry from a registered wearable device associated with a user.
Generating proximity event records comprising device identifier data and timestamps for at least one detected wearable device.
Computing at least one cross-device similarity metric by comparing movement-related sensor outputs across the registered wearable device and at least one detected wearable device.
Generating a fraud risk score based at least in part on the at least one cross-device similarity metric and a proximity recurrence measure.
Outputting a cryptographically verifiable activity certificate when the fraud risk score indicates legitimacy.
Suppressing issuance of the activity certificate and generating a fraud alert when the fraud risk score indicates fraud.

Further, the proximity module detects the one or more additional wearable devices using Bluetooth Low Energy signals and estimates distance based on received signal characteristics.

Further, the proximity module further detects the one or more additional wearable devices using ultra-wideband ranging based on time-of-flight measurements.

Further, the feature extraction module derives motion features comprising cadence, step timing, and accelerometer-based gait characteristics.

Further, the cross-device analysis module performs temporal alignment of motion features prior to computing the one or more similarity metrics.

Further, the decision module incorporates a user-specific historical baseline and detects deviations from the baseline indicative of anomalous activity.

Further, the fraud risk score is generated using a machine learning model trained to classify activity as legitimate or non-legitimate based on multi-device sensor data.

Further, the machine learning model comprises an ensemble of classifiers whose outputs are combined using weighted aggregation.

Further, the proximity event records are stored in the tamper-evident log using cryptographic hash chaining.

Further, the certificate module generates the cryptographically verifiable activity certificate using public-key cryptography.

Further, the cryptographically verifiable activity certificate comprises an identifier of the user, a verified activity time period, and a digital signature.

Further, the system constructs a device relationship graph comprising nodes representing wearable devices and edges representing proximity relationships.

Further, the cross-device analysis module applies graph analysis techniques to identify clusters of devices exhibiting repeated co-location.

Further, the system further generates audit records identifying data inputs and decision factors used to generate the fraud risk score.

Further, the system suppresses issuance of the cryptographically verifiable activity certificate upon detection of synchronized motion patterns across multiple devices.

Further, the device identifiers included in the proximity event records are stored in hashed or pseudonymous form.

Further, the system supports data collection from heterogeneous wearable devices and normalizes device-specific data into a standardized format.

Further, the system maintains a certificate revocation list and invalidates previously issued certificates upon subsequent detection of fraudulent activity.

In some embodiments, the present disclosure describes a system and method for verifying physical activity recorded by wearable devices. The system detects multiple wearable devices in proximity using wireless communication signals and compares sensor data, including accelerometer patterns, step counts, and heart rate, to identify synchronized motion indicative of multi-device activity. Machine-learning models incorporating ensemble classifiers, behavioral biometrics, and adaptive thresholds generate fraud risk scores. When activity satisfies verification criteria, the system produces cryptographically signed certificates, optionally with blockchain anchoring. The platform includes mobile applications, backend services with real-time fraud detection, integration with heterogeneous wearable devices, and APIs for third-party systems, providing audit-grade activity verification data suitable for downstream processing.

In some embodiments, the disclosed system may provide a technical improvement to the field of physical activity verification by addressing the technical problem that conventional activity tracking systems rely on isolated device telemetry that is susceptible to manipulation, duplication, and fraudulent replication. In such embodiments, the system may improve wearable data verification technology by correlating motion data with proximity data obtained from multiple wearable devices, thereby enabling detection of synchronized movement patterns that are not observable through single-device analysis. The said correlation-based verification may be implemented by aligning accelerometer-derived motion vectors from different devices and computing similarity metrics across synchronized time windows, which may include dynamic time warping, cross-correlation functions, or frequency-domain similarity measures. By performing such correlation at a server-side processing device, the system may improve the robustness of activity verification platforms against multi-device fraud scenarios, thereby enhancing the reliability of incentive-based health technologies.

In some embodiments, the disclosed system may improve proximity detection technology by addressing the technical problem that transient wireless encounters may generate false co-location signals. In such embodiments, the system may implement proximity persistence evaluation by aggregating proximity data over successive time intervals and computing a continuity measure that reflects sustained spatial co-location rather than momentary signal overlap. Which may be achieved, for example, by maintaining rolling windows of received signal strength indicator values or time-of-flight measurements and filtering out proximity events that do not satisfy temporal stability criteria. As a result, the proximity detection subsystem may be technically improved to distinguish meaningful co-location indicative of potential device stacking from incidental proximity encounters, thereby increasing accuracy in fraud detection systems that rely on wireless signal analysis.

In some embodiments, the disclosed system may improve time-series data processing technology by addressing the technical problem of temporal drift and misalignment between heterogeneous wearable devices. In such embodiments, the system may normalize timestamp data associated with motion telemetry and proximity events using server-side clock reconciliation techniques, interpolation, or resampling methods. For example, motion data streams sampled at different frequencies may be resampled to a common temporal resolution, and timestamp offsets may be corrected using synchronization markers derived from proximity detection events. The said temporal alignment may enable accurate computation of motion similarity metrics across devices, thereby improving multi-source sensor fusion technology used in distributed activity verification systems.

In some embodiments, the disclosed system may improve fraud detection technology by addressing the technical problem that binary classification of activity as legitimate or fraudulent lacks granularity and adaptability. In such embodiments, the system may generate a fraud risk score derived from weighted combinations of motion similarity metrics, proximity persistence values, and historical deviation measures. The weighting may be dynamically adjusted based on user-specific baselines or program-specific policies stored in a storage device. The given risk-based scoring approach may improve machine-assisted decision systems by enabling adaptive thresholds, graduated responses, and contextual interpretation of activity authenticity, thereby enhancing the technical effectiveness of automated fraud analysis engines.

In some embodiments, the disclosed system may improve auditability and compliance technology by addressing the technical problem that conventional activity tracking systems lack verifiable decision provenance. In such embodiments, the system may generate audit data objects that encapsulate motion similarity metrics, fraud risk scores, threshold parameters, and verification outcomes, and may store such audit data objects in tamper-evident data structures using cryptographic hash chaining or append-only logs. The given implementation may improve data integrity and audit trail technologies by enabling post hoc verification of activity validation decisions without reliance on mutable operational databases.

In some embodiments, the disclosed system may improve interoperability technology by addressing the technical problem that downstream systems consume verification results in heterogeneous formats. In such embodiments, the system may encode verification status data into a predefined schema that enforces structural consistency and semantic validation prior to storage or transmission. Schema validation may be performed at the processing device using predefined constraints, thereby improving data exchange technology by reducing integration errors and enabling reliable consumption of verification outputs by external systems such as reward engines or compliance platforms.

In some embodiments, the disclosed system may further improve proximity analysis technology by incorporating adaptive proximity thresholding to address the technical problem that static distance thresholds fail to account for environmental variability. In such embodiments, the system may dynamically adjust proximity range thresholds based on signal variance, device type metadata, or historical proximity noise profiles stored in the storage device. For example, proximity thresholds may be tightened in environments exhibiting stable signal characteristics and relaxed in environments with known multipath interference. The given adaptive approach may improve wireless signal interpretation technology by reducing false positives in proximity-based fraud detection.

In some embodiments, the disclosed system may further improve motion analysis technology by incorporating frequency-domain feature extraction to address the technical problem that time-domain similarity metrics may be sensitive to noise and transient perturbations. In such embodiments, the processing device may transform motion data into spectral representations using techniques such as fast Fourier transforms or wavelet decompositions and may compare spectral energy distributions across devices. The given implementation may improve signal processing technology by enabling detection of mechanically induced synchronized motion that exhibits distinct frequency signatures compared to human gait patterns.

In some embodiments, the disclosed system may further improve personalization technology by introducing adaptive historical baselines that evolve over time to address the technical problem that static user profiles become stale. In such embodiments, the system may update historical activity profiles using decay functions or weighted averaging that emphasize recent verified activity while retaining long-term behavioral characteristics. The given approach may improve behavioral modeling technology by allowing fraud detection logic to remain sensitive to gradual lifestyle changes without increasing false positives.

In some embodiments, the disclosed system may further improve scalability technology by enabling batch-oriented verification pipelines that process aggregated activity and proximity data asynchronously. In such embodiments, the storage device may buffer intermediate verification data, and the processing device may perform batch correlation analysis during off-peak intervals. The given implementation may improve distributed computing technology by reducing real-time processing load while preserving verification accuracy in large-scale deployments.

In some embodiments, the disclosed system may further improve security technology by introducing verification output sealing mechanisms that cryptographically bind verification status data to underlying audit data objects. In such embodiments, the processing device may compute a cryptographic digest over the verification status data and associated audit data and store the digest in the storage device for later validation, improving data authenticity technology by ensuring that verification results may not be altered without detection.

In some embodiments, the disclosed system may further improve policy enforcement technology by enabling rule-based verification modulation, wherein verification criteria may be selected or modified based on program identifiers or regulatory requirements stored in the storage device. In such embodiments, the processing device may select different similarity thresholds, persistence requirements, or scoring weights depending on the applicable policy context, improving configurable compliance technology by allowing a single verification platform to support diverse operational requirements without architectural modification.

In some embodiments, a wearable device system is and/or includes a plurality of wearable devices, and is configured to generate sensor data representing user motion and contextual conditions, and is configured to communicate the sensor data over a communication network to a computing system. The wearable device system may include at least a first wearable device (i.e., wearable device system) worn by a first user and a second wearable device (i.e., another wearable device) worn by a second user, wherein the first wearable device is configured to detect the second wearable device within a proximity range and to generate proximity data indicative of such detection.

Each wearable device and/or the wearable device system may be and/or may include a multi-sensor system. Further, the multi-sensor system may be and/or may include a plurality of sensors of different modalities. By way of example and without limitation, the multi-sensor system may be and/or may include an inertial measurement unit (IMU) including one or more accelerometers and gyroscopes, a magnetometer, a barometer, a GNSS receiver, an optical sensor (e.g., photoplethysmography (PPG)), and/or a skin-contact or temperature sensor. In certain embodiments, at least a subset of the sensor data is sampled at a first rate suitable for motion characterization (e.g., IMU sampling) and a second subset is sampled at a second rate suitable for physiological or contextual characterization (e.g., PPG, temperature). In some embodiments, the wearable device system generates activity data comprising the sensor data. The activity data may include raw sensor samples, filtered sensor signals, derived motion features (e.g., step cadence, stride periodicity, orientation changes, jerk, spectral components, and/or gait signatures), and/or a time series of feature vectors. The activity data may further include timestamps generated by a wearable clock and/or a synchronized clock reference. The wearable device system may also generate proximity data indicative of detection of another wearable device within a proximity range. In some embodiments, the proximity data is derived from short-range wireless signaling using one or more proximity technologies, including Bluetooth Low Energy (BLE), ultra-wideband (UWB), near-field communication (NFC), or Wi-Fi awareness. The proximity data may include a received signal strength indicator (RSSI), time-of-flight measurements, channel impulse response information, and/or an estimated distance and confidence value. In certain embodiments, the proximity range is determined dynamically based on environmental calibration, device orientation, and/or multipath mitigation. The processing device may extract motion features from the activity data using windowing and feature extraction operations, and may compute a motion similarity metric representing synchronized movement between the wearable device system and the another wearable device system (i.e., another wearable device). The motion similarity metric may be determined using cross-correlation, dynamic time warping (DTW), frequency-domain similarity, and/or state-estimation-based alignment, optionally producing a confidence value reflecting sensor quality and proximity confidence.

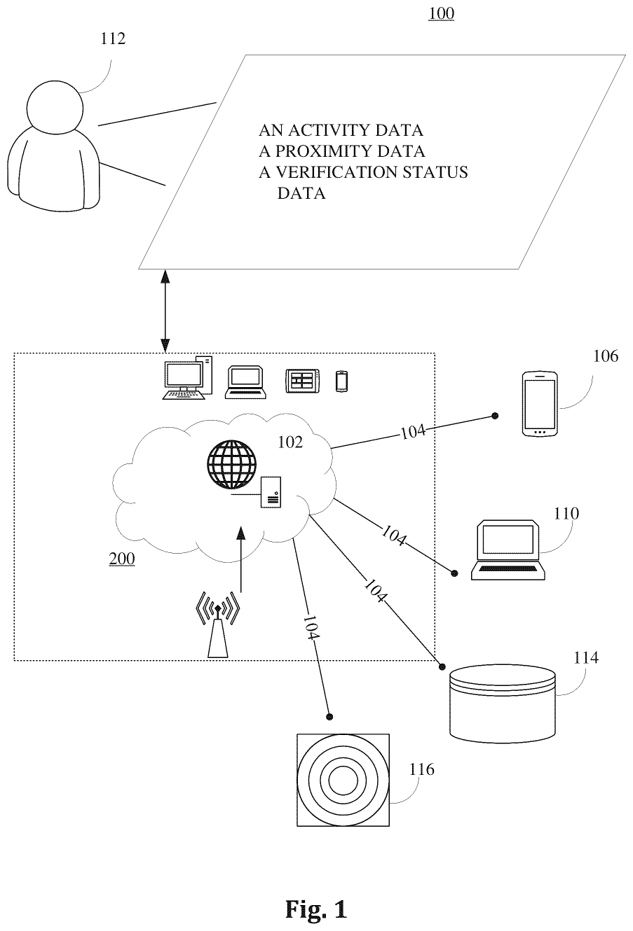

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

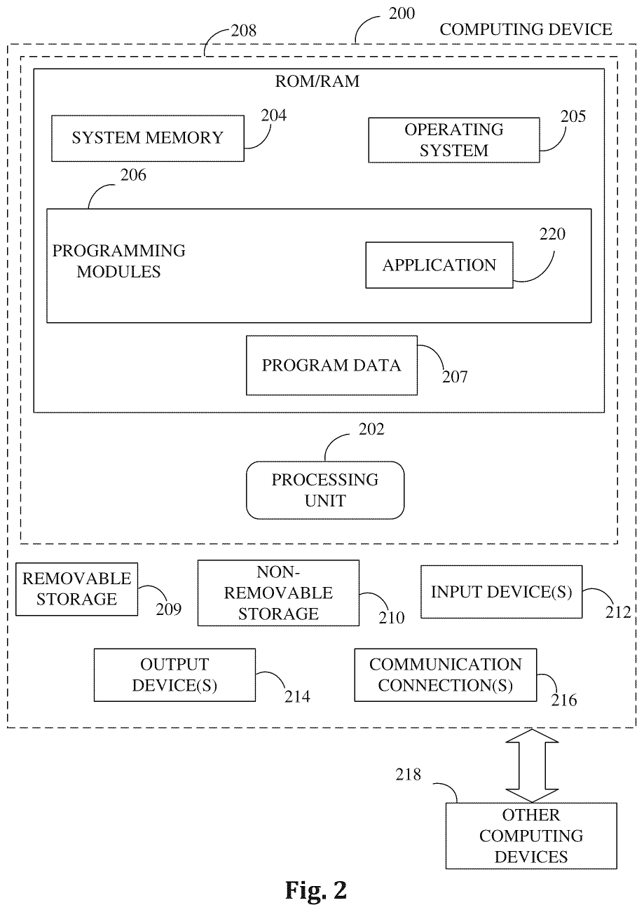

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM,

digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods'stages may be modified in any manner, including by reordering stages and/or inserting or deleting stages, without departing from the disclosure.

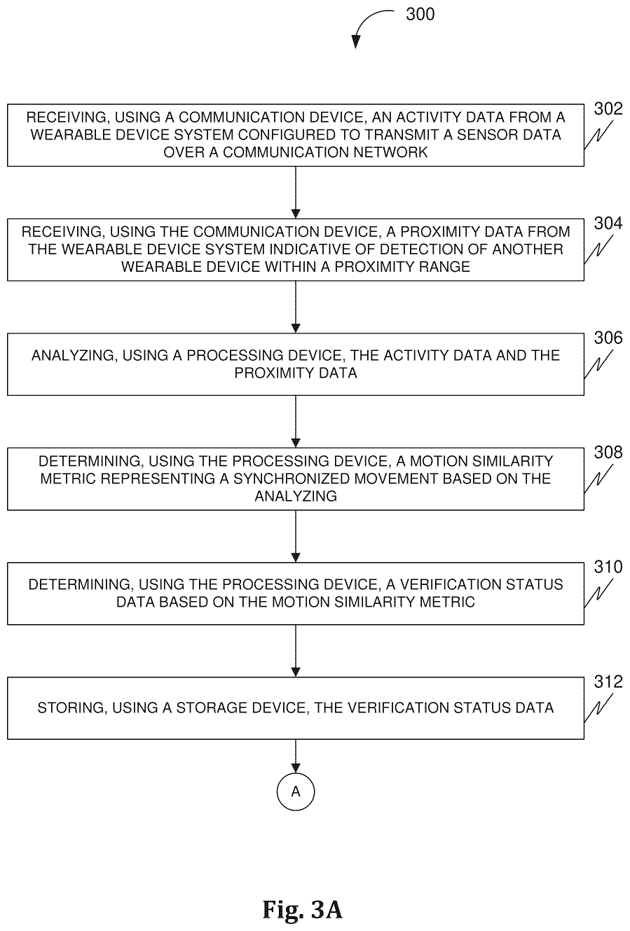

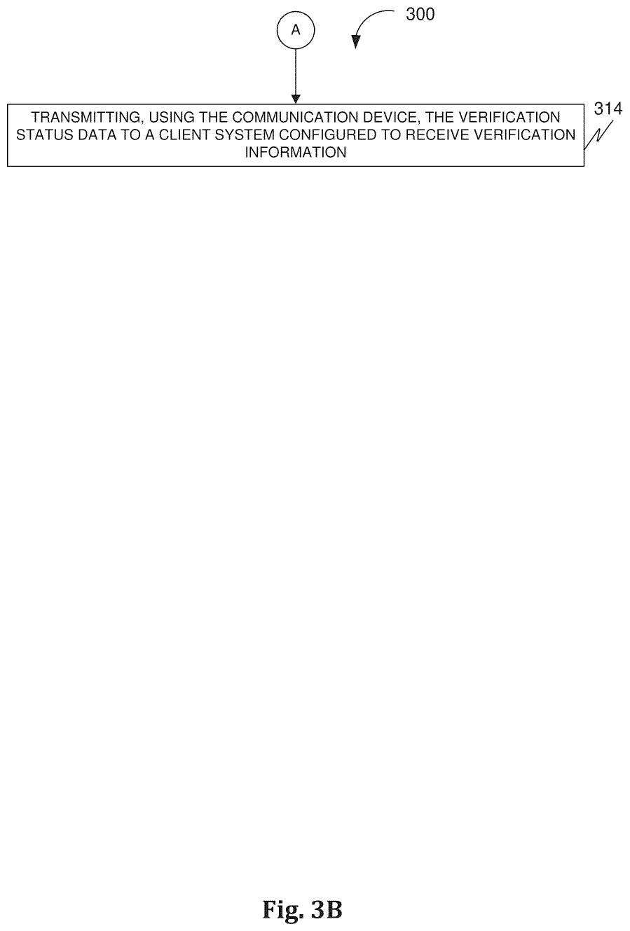

FIG. 3A and FIG. 3B illustrate a flowchart of a method 300 of verifying a physical activity, in accordance with some embodiments. Accordingly, the method 300 may include a step 302 of receiving, using a communication device 1302, an activity data from a wearable device system which may be configured to transmit a sensor data over a communication network. Further, the method 300 may include a step 304 of receiving, using the communication device 1302, a proximity data from the wearable device system indicative of detection of another wearable device within a proximity range. Further, the method 300 may include a step 306 of analyzing, using a processing device 1304, the activity data and the proximity data. Further, the method 300 may include a step 308 of determining, using the processing device 1304, a motion similarity metric representing a synchronized movement based on the analyzing. Further, the method 300 may include a step 310 of determining, using the processing device 1304, a verification status data based on the motion similarity metric. Further, the method 300 may include a step 312 of storing, using a storage device 1306, the verification status data. Further, the method 300 may include a step 314 of transmitting, using the communication device 1302, the verification status data to a client system which may be configured to receive verification information. Further, the activity data represents and/or characterizes the physical activity. Further, the activity data includes the sensor data. Further, the wearable device system may be configured for recording the physical activity and generating the sensor data based on the recording. Further, the physical activity may include a physical activity of the wearable device system, and a physical activity of a user wearing the wearable device system. Further, the physical activity may be and/or may include an activity.

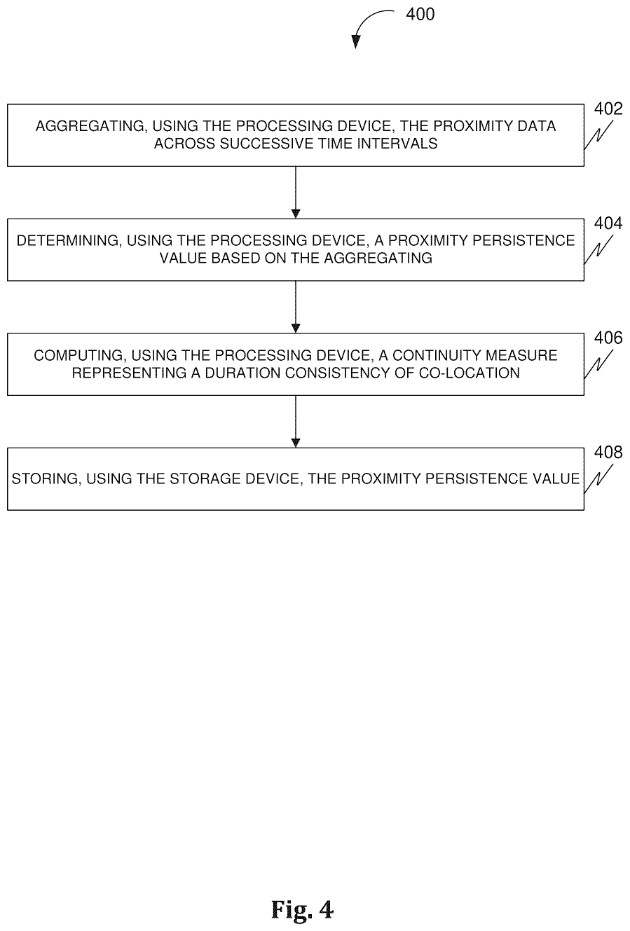

FIG. 4 illustrates a flowchart of a method 400 of verifying a physical activity including computing, using the processing device 1304, a continuity measure representing a duration consistency of co-location, in accordance with some embodiments. Further, in some embodiments, the method 400 further may include a step 402 of aggregating, using the processing device 1304, the proximity data across successive time intervals. Further, in some embodiments, the method 400 further may include a step 404 of determining, using the processing device 1304, a proximity persistence value based on the aggregating. Further, in some embodiments, the method 400 further may include a step 406 of computing, using the processing device 1304, a continuity measure representing a duration consistency of co-location. Further, in some embodiments, the method 400 further may include a step 408 of storing, using the storage device 1306, the proximity persistence value.

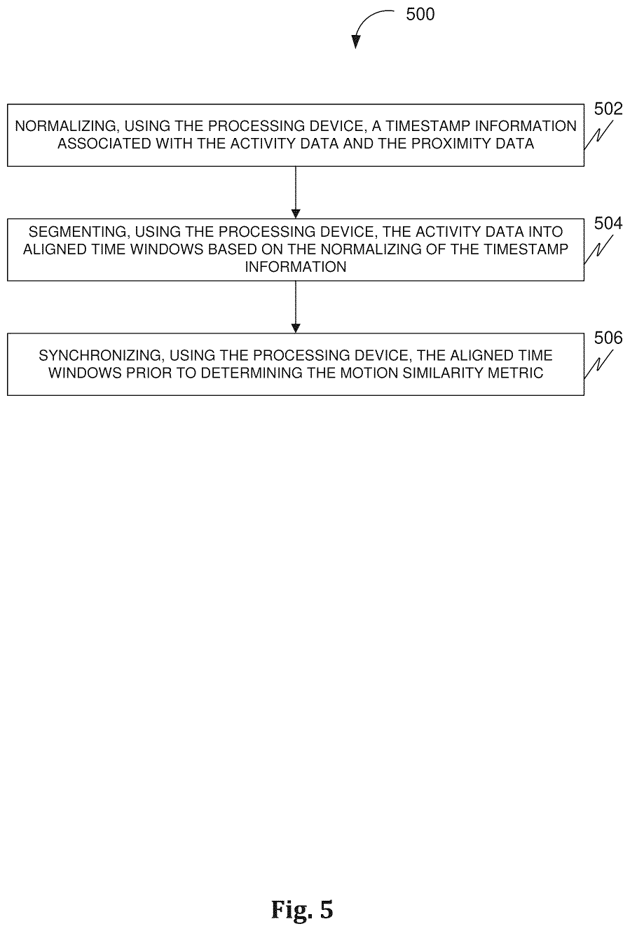

FIG. 5 illustrates a flowchart of a method 500 of verifying a physical activity including synchronizing, using the processing device 1304, the aligned time windows prior to determining the motion similarity metric, in accordance with some embodiments. Further, in some embodiments, the method 500 further may include a step 502 of normalizing, using the processing device 1304, a timestamp information associated with the activity data and the proximity data. Further, in some embodiments, the method 500 further may include a step 504 of segmenting, using the processing device 1304, the activity data into aligned time windows based on the normalizing of the timestamp information. Further, in some embodiments, the method 500 further may include a step 506 of synchronizing, using the processing device 1304, the aligned time windows prior to determining the motion similarity metric.

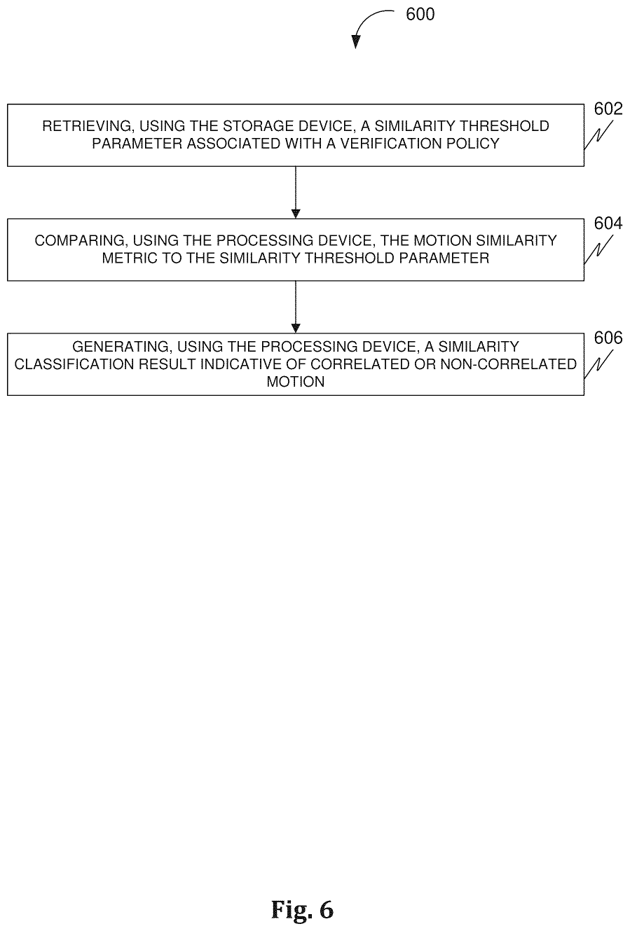

FIG. 6 illustrates a flowchart of a method 600 of verifying a physical activity including generating, using the processing device 1304, a similarity classification result indicative of correlated or non-correlated motion, in accordance with some embodiments. Further, in some embodiments, the method 600 further may include a step 602 of retrieving, using the storage device 1306, a similarity threshold parameter associated with a verification policy. Further, in some embodiments, the method 600 further may include a step 604 of comparing, using the processing device 1304, the motion similarity metric to the similarity threshold parameter. Further, in some embodiments, the method 600 further may include a step 606 of generating, using the processing device 1304, a similarity classification result indicative of correlated or non-correlated motion.

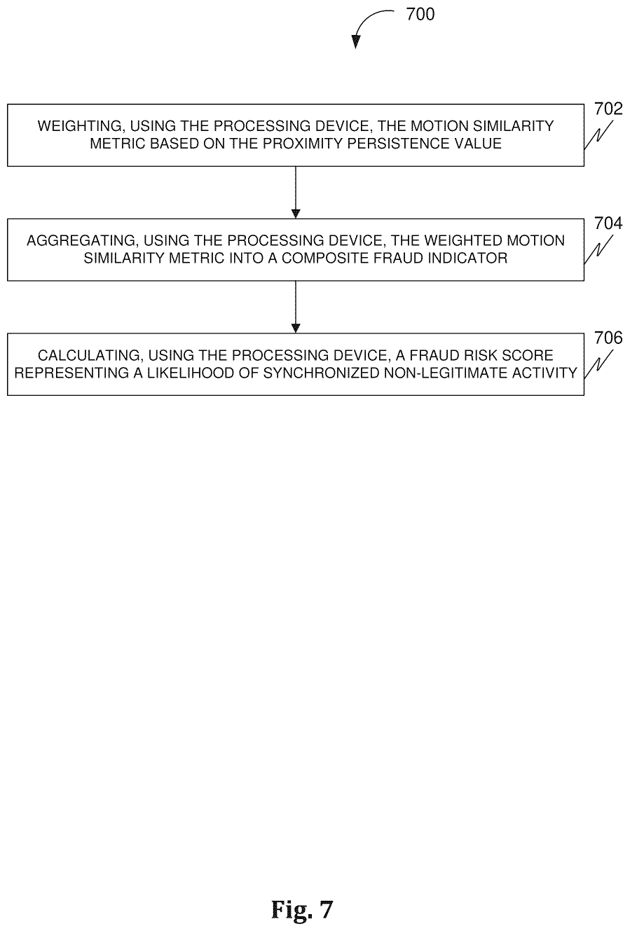

FIG. 7 illustrates a flowchart of a method 700 of verifying a physical activity including calculating, using the processing device 1304, a fraud risk score representing a likelihood of synchronized non-legitimate activity, in accordance with some embodiments. Further, in some embodiments, the method 700 further may include a step 702 of weighting, using the processing device 1304, the motion similarity metric based on the proximity persistence value. Further, in some embodiments, the method 700 further may include a step 704 of aggregating, using the processing device 1304, the weighted motion similarity metric into a composite fraud indicator. Further, in some embodiments, the method 700 further may include a step 706 of calculating, using the processing device 1304, a fraud risk score representing a likelihood of synchronized non-legitimate activity.

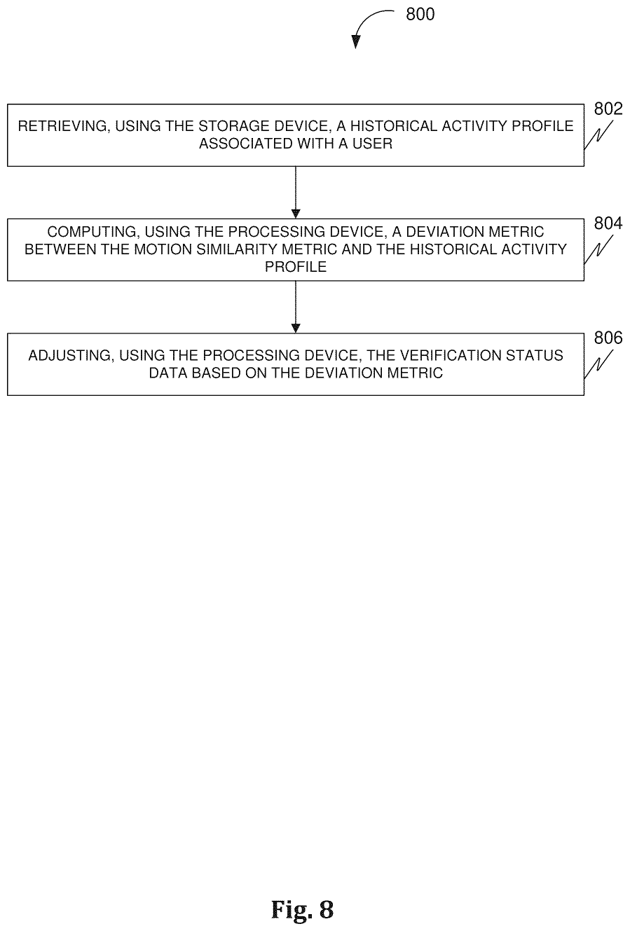

FIG. 8 illustrates a flowchart of a method 800 of verifying a physical activity including adjusting, using the processing device 1304, the verification status data, in accordance with some embodiments. Further, in some embodiments, the method 800 further may include a step 802 of retrieving, using the storage device 1306, a historical activity profile associated with a user. Further, in some embodiments, the method 800 further may include a step 804 of computing, using the processing device 1304, a deviation metric between the motion similarity metric and the historical activity profile. Further, in some embodiments, the method 800 further may include a step 806 of adjusting, using the processing device 1304, the verification status data based on the deviation metric.

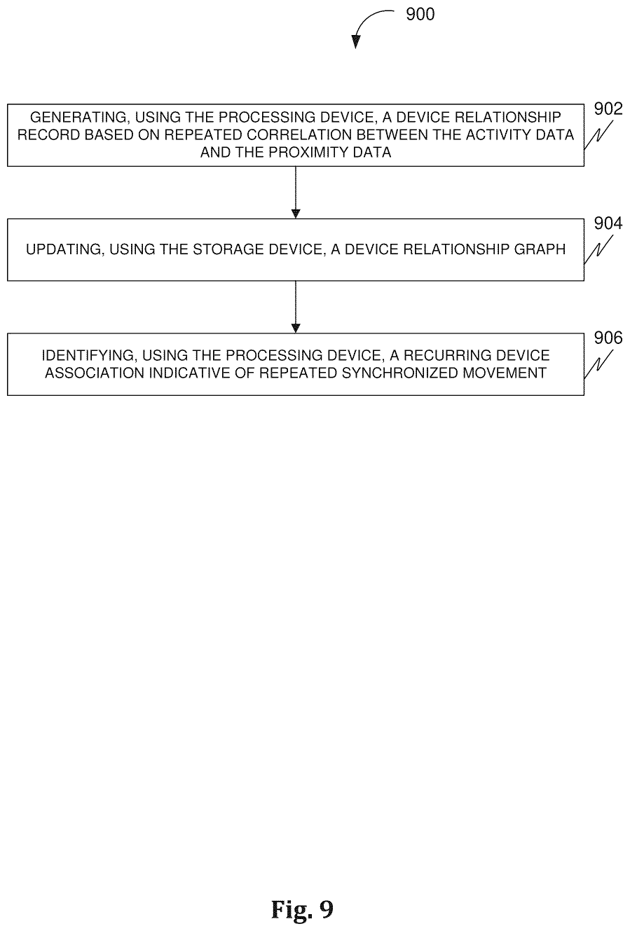

FIG. 9 illustrates a flowchart of a method 900 of verifying a physical activity including identifying, using the processing device 1304, a recurring device association indicative of repeated synchronized movement, in accordance with some embodiments. Further, in some embodiments, the method 900 further may include a step 902 of generating, using the processing device 1304, a device relationship record based on repeated correlation between the activity data and the proximity data. Further, in some embodiments, the method 900 further may include a step 904 of updating, using the storage device 1306, a device relationship graph. Further, in some embodiments, the method 900 further may include a step 906 of identifying, using the processing device 1304, a recurring device association indicative of repeated synchronized movement.

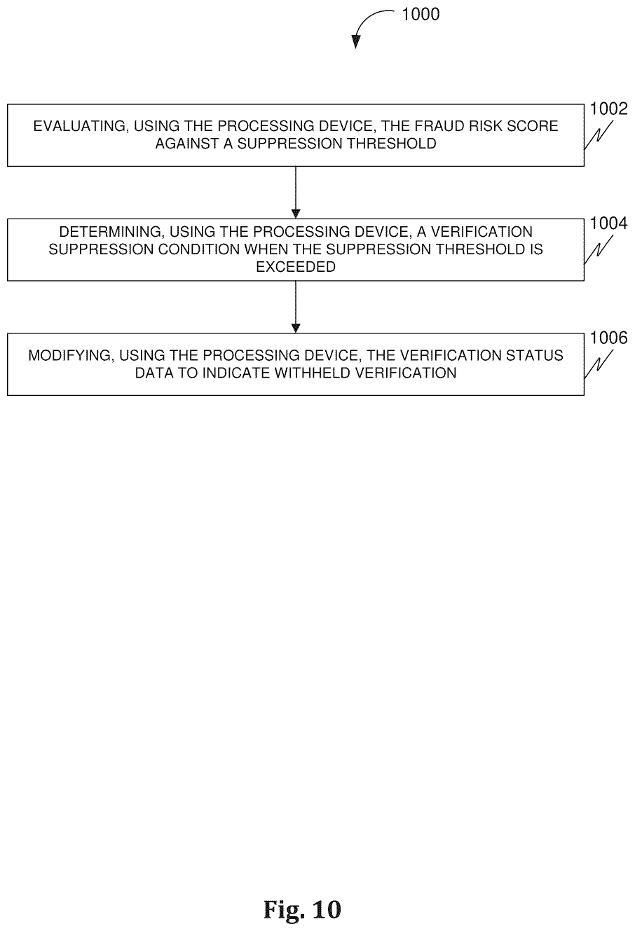

FIG. 10 illustrates a flowchart of a method 1000 of verifying a physical activity including modifying, using the processing device 1304, the verification status data to indicate withheld verification, in accordance with some embodiments. Further, in some embodiments, the method 1000 further may include a step 1002 of evaluating, using the processing device 1304, the fraud risk score against a suppression threshold. Further, in some embodiments, the method 1000 further may include a step 1004 of determining, using the processing device 1304, a verification suppression condition when the suppression threshold may be exceeded. Further, in some embodiments, the method 1000 further may include a step 1006 of modifying, using the processing device 1304, the verification status data to indicate withheld verification.

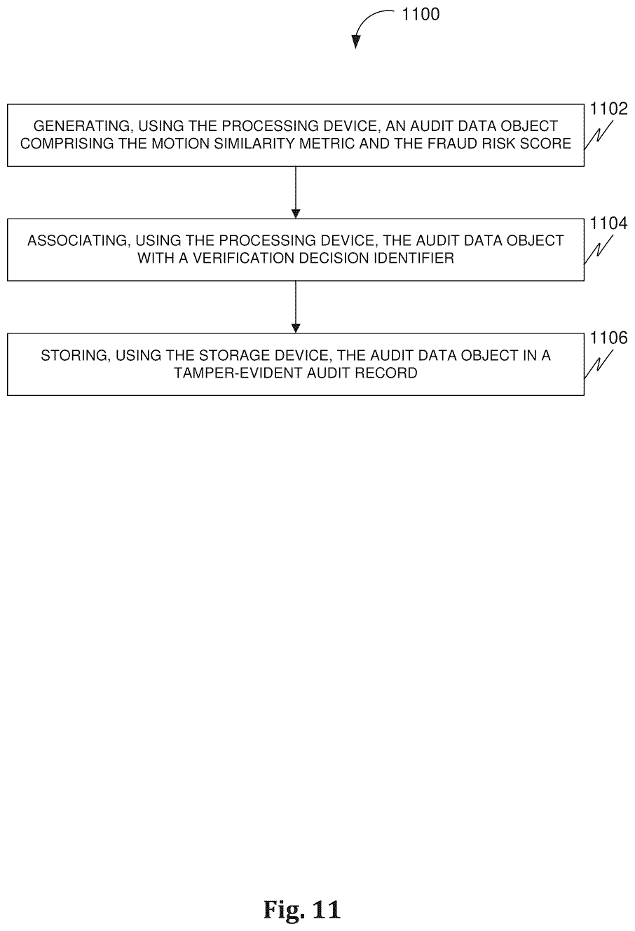

FIG. 11 illustrates a flowchart of a method 1100 of verifying a physical activity including associating, using the processing device 1304, the audit data object with a verification decision identifier, in accordance with some embodiments. Further, in some embodiments, the method 1100 further may include a step 1102 of generating, using the processing device 1304, an audit data object comprising the motion similarity metric and the fraud risk score. Further, in some embodiments, the method 1100 further may include a step 1104 of associating, using the processing device 1304, the audit data object with a verification decision identifier. Further, in some embodiments, the method 1100 further may include a step 1106 of storing, using the storage device 1306, the audit data object in a tamper-evident audit record.

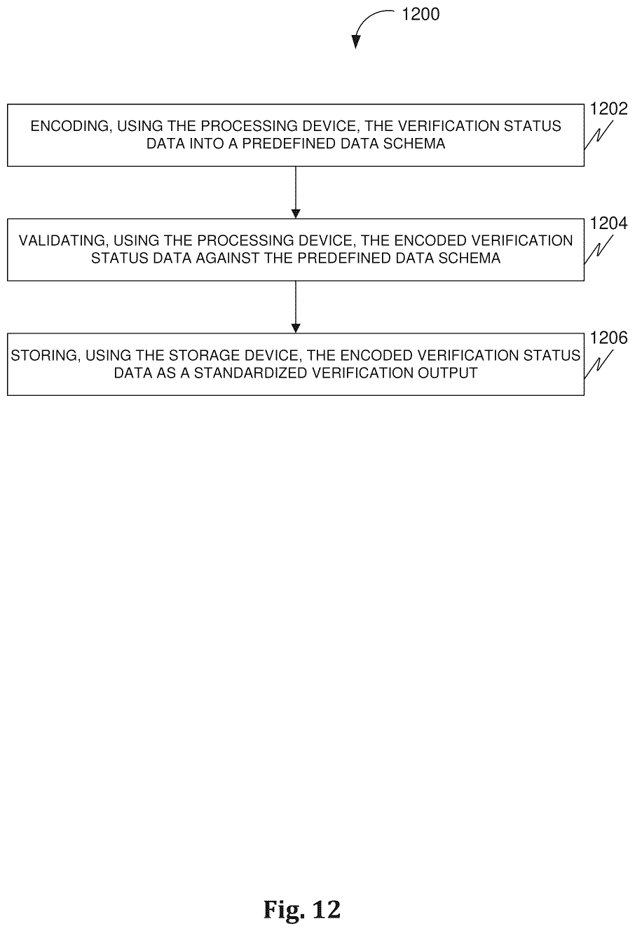

FIG. 12 illustrates a flowchart of a method 1200 of verifying a physical activity including validating, using the processing device 1304, the encoded verification status data against the predefined data schema, in accordance with some embodiments. Further, in some embodiments, the method 1200 further may include a step 1202 of encoding, using the processing device 1304, the verification status data into a predefined data schema. Further, in some embodiments, the method 1200 further may include a step 1204 of validating, using the processing device 1304, the encoded verification status data against the predefined data schema. Further, in some embodiments, the method 1200 further may include a step 1206 of storing, using the storage device 1306, the encoded verification status data as a standardized verification output.

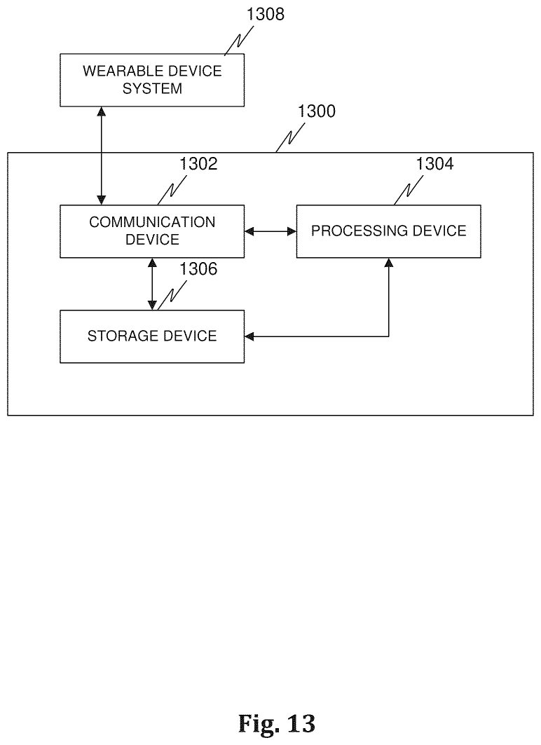

FIG. 13 illustrates a block diagram of a system 1300 for verifying a physical activity, in accordance with some embodiments. Accordingly, the system 1300 may include a communication device 1302. Further, the communication device 1302 may be configured for receiving an activity data from a wearable device system 1308 which may be configured to transmit a sensor data over a communication network. Further, the communication device 1302 may be configured for receiving a proximity data from the wearable device system 1308 indicative of detection of another wearable device within a proximity range. Further, the communication device 1302 may be configured for transmitting a verification status data to a client system which may be configured to receive verification information. Further, the system 1300 may include a processing device 1304 communicatively coupled with the communication device 1302. Further, the processing device 1304 may be configured for analyzing the activity data and the proximity data. Further, the processing device 1304 may be configured for determining a motion similarity metric representing a synchronized movement based on the analyzing. Further, the processing device 1304 may be configured for determining the verification status data based on the motion similarity metric. Further, the system 1300 may include a storage device 1306 communicatively coupled with the processing device 1304. Further, the storage device 1306 may be configured for storing the verification status data. Further, the wearable device system 1308 may be and/or may include a sensor, a plurality of sensors, a multi sensor system, a user device, a registered user device, a client device, a computing device, etc.

Further, in some embodiments, the processing device 1304 may be further configured for aggregating the proximity data across successive time intervals. Further, the processing device 1304 may be further configured for determining a proximity persistence value based on the aggregating. Further, the processing device 1304 may be further configured for computing a continuity measure representing a duration consistency of co-location. Further, the storage device 1306 may be further configured for storing the proximity persistence value.

Further, in some embodiments, the processing device 1304 may be further configured for normalizing a timestamp information associated with the activity data and the proximity data. Further, the processing device 1304 may be further configured for segmenting the activity data into aligned time windows based on the normalizing of the timestamp information. Further, the processing device 1304 may be further configured for synchronizing the aligned time windows prior to determining the motion similarity metric.

Further, in some embodiments, the storage device 1306 may be further configured for retrieving a similarity threshold parameter associated with a verification policy. Further, the processing device 1304 may be further configured for comparing the motion similarity metric to the similarity threshold parameter. Further, the processing device 1304 may be further configured for generating a similarity classification result indicative of correlated or non-correlated motion.

Further, in some embodiments, the processing device 1304 may be further configured for weighting the motion similarity metric based on the proximity persistence value. Further, the processing device 1304 may be further configured for aggregating the weighted motion similarity metric into a composite fraud indicator. Further, the processing device 1304 may be further configured for calculating a fraud risk score representing a likelihood of synchronized non-legitimate activity.

Further, in some embodiments, the storage device 1306 may be further configured for retrieving a historical activity profile associated with a user. Further, the processing device 1304 may be further configured for computing a deviation metric between the motion similarity metric and the historical activity profile. Further, the processing device 1304 may be further configured for adjusting the verification status data based on the deviation metric.

Further, in some embodiments, the processing device 1304 may be further configured for generating a device relationship record based on repeated correlation between the activity data and the proximity data. Further, the processing device 1304 may be further configured for identifying a recurring device association indicative of repeated synchronized movement. Further, the storage device 1306 may be further configured for updating a device relationship graph.

Further, in some embodiments, the processing device 1304 may be further configured for evaluating the fraud risk score against a suppression threshold. Further, the processing device 1304 may be further configured for determining a verification suppression condition when the suppression threshold may be exceeded. Further, the processing device 1304 may be further configured for modifying the verification status data to indicate withheld verification.

Further, in some embodiments, the processing device 1304 may be further configured for generating an audit data object comprising the motion similarity metric and the fraud risk score. Further, the processing device 1304 may be further configured for associating the audit data object with a verification decision identifier. Further, the storage device 1306 may be further configured for storing the audit data object in a tamper-evident audit record.

Further, in some embodiments, the processing device 1304 may be further configured for encoding the verification status data into a predefined data schema. Further, the processing device 1304 may be further configured for validating the encoded verification status data against the predefined data schema. Further, the storage device 1306 may be further configured for storing the encoded verification status data as a standardized verification output.

Although the invention has been explained in relation to its preferred embodiment, it is to be understood that many other possible modifications and variations can be made without departing from the spirit and scope of the invention as hereinafter claimed.

What is claimed is:

1. A method of verifying a physical activity, the method comprising:
receiving, using a communication device, an activity data from a wearable device system configured to transmit a sensor data over a communication network;
receiving, using the communication device, a proximity data from the wearable device system indicative of detection of another wearable device within a proximity range;
analyzing, using a processing device, the activity data and the proximity data;
determining, using the processing device, a motion similarity metric representing a synchronized movement based on the analyzing;
determining, using the processing device, a verification status data based on the motion similarity metric;
storing, using a storage device, the verification status data; and
transmitting, using the communication device, the verification status data to a client system configured to receive verification information.

2. The method of claim 1 further comprising:
aggregating, using the processing device, the proximity data across successive time intervals;
determining, using the processing device, a proximity persistence value based on the aggregating;
computing, using the processing device, a continuity measure representing a duration consistency of co-location; and
storing, using the storage device, the proximity persistence value.

3. The method of claim 1 further comprising:
normalizing, using the processing device, a timestamp information associated with the activity data and the proximity data;
segmenting, using the processing device, the activity data into aligned time windows based on the normalizing of the timestamp information; and
synchronizing, using the processing device, the aligned time windows prior to determining the motion similarity metric.

4. The method of claim 1 further comprising:
retrieving, using the storage device, a similarity threshold parameter associated with a verification policy;
comparing, using the processing device, the motion similarity metric to the similarity threshold parameter; and
generating, using the processing device, a similarity classification result indicative of correlated or non-correlated motion.

5. The method of claim 1 further comprising:
weighting, using the processing device, the motion similarity metric based on the proximity persistence value;
aggregating, using the processing device, the weighted motion similarity metric into a composite fraud indicator; and
calculating, using the processing device, a fraud risk score representing a likelihood of synchronized non-legitimate activity.

6. The method of claim 1 further comprising:
retrieving, using the storage device, a historical activity profile associated with a user;
computing, using the processing device, a deviation metric between the motion similarity metric and the historical activity profile; and
adjusting, using the processing device, the verification status data based on the deviation metric.

7. The method of claim 1 further comprising:
generating, using the processing device, a device relationship record based on repeated correlation between the activity data and the proximity data;
updating, using the storage device, a device relationship graph; and
identifying, using the processing device, a recurring device association indicative of repeated synchronized movement.

8. The method of claim 1 further comprising:
evaluating, using the processing device, the fraud risk score against a suppression threshold;
determining, using the processing device, a verification suppression condition when the suppression threshold is exceeded; and
modifying, using the processing device, the verification status data to indicate withheld verification.

9. The method of claim 1 further comprising:
generating, using the processing device, an audit data object comprising the motion similarity metric and the fraud risk score;
associating, using the processing device, the audit data object with a verification decision identifier; and
storing, using the storage device, the audit data object in a tamper-evident audit record.

10. The method of claim 1 further comprising:
encoding, using the processing device, the verification status data into a predefined data schema;
validating, using the processing device, the encoded verification status data against the predefined data schema; and
storing, using the storage device, the encoded verification status data as a standardized verification output.

11. A system for verifying a physical activity, the system comprising:
a communication device configured for:
receiving an activity data from a wearable device system configured to transmit a sensor data over a communication network;
receiving a proximity data from the wearable device system indicative of detection of another wearable device within a proximity range; and
transmitting a verification status data to a client system configured to receive verification information;
a processing device communicatively coupled with the communication device, wherein the processing device is configured for:
analyzing the activity data and the proximity data;
determining a motion similarity metric representing a synchronized movement based on the analyzing; and
determining the verification status data based on the motion similarity metric; and
a storage device communicatively coupled with the processing device, wherein the storage device is configured for storing the verification status data.

12. The system of claim 11, wherein the processing device is further configured for:
aggregating the proximity data across successive time intervals;
determining a proximity persistence value based on the aggregating; and
computing a continuity measure representing a duration consistency of co-location, wherein the storage device is further configured for storing the proximity persistence value.

13. The system of claim 11, wherein the processing device is further configured for:
normalizing a timestamp information associated with the activity data and the proximity data;
segmenting the activity data into aligned time windows based on the normalizing of the timestamp information; and
synchronizing the aligned time windows prior to determining the motion similarity metric.

14. The system of claim 11, wherein the storage device is further configured for retrieving a similarity threshold parameter associated with a verification policy, wherein the processing device is further configured for:
comparing the motion similarity metric to the similarity threshold parameter; and
generating a similarity classification result indicative of correlated or non-correlated motion.

15. The system of claim 11, wherein the processing device is further configured for:
weighting the motion similarity metric based on the proximity persistence value;
aggregating the weighted motion similarity metric into a composite fraud indicator; and
calculating a fraud risk score representing a likelihood of synchronized non-legitimate activity.

16. The system of claim 11, wherein the storage device is further configured for retrieving a historical activity profile associated with a user, wherein the processing device is further configured for:
computing a deviation metric between the motion similarity metric and the historical activity profile; and
adjusting the verification status data based on the deviation metric.

17. The system of claim 11, wherein the processing device is further configured for:
generating a device relationship record based on repeated correlation between the activity data and the proximity data; and
identifying a recurring device association indicative of repeated synchronized movement, wherein the storage device is further configured for updating a device relationship graph.

18. The system of claim 11, wherein the processing device is further configured for:
evaluating the fraud risk score against a suppression threshold;
determining a verification suppression condition when the suppression threshold is exceeded; and
modifying the verification status data to indicate withheld verification.

19. The system of claim 11, wherein the processing device is further configured for:
generating an audit data object comprising the motion similarity metric and the fraud risk score; and
associating the audit data object with a verification decision identifier, wherein the storage device is further configured for storing the audit data object in a tamper-evident audit record.

20. The system of claim 11, wherein the processing device is further configured for:
encoding the verification status data into a predefined data schema; and
validating the encoded verification status data against the predefined data schema, wherein the storage device is further configured for storing the encoded verification status data as a standardized verification output.
